(19) United States
(12) Patent Application Publication
(10) Pub. No.: US 2020/0289660 A1
(43) Pub. Date: Sep. 17, 2020

(54) PYRROLOBENZODIAZEPINE CONJUGATES

(71) Applicant: MEDIMMUNE LIMITED, Cambridge, Cambridgeshire (GB)

(72) Inventors: Philip Wilson HOWARD, Cambridge, Cambridgeshire (GB); Luke MASTERSON, Cambridge, Cambridgeshire (GB); Thais CAILLEAU, Cambridge, Cambridgeshire (GB); Nazzareno DIMASI, Gaithersburg, MD (US); Jason WHITE, Gaithersburg, MD (US)

(21) Appl. No.: 16/763,814

(22) PCT Filed: Nov. 13, 2018
(86) PCT No.: PCT/EP2018/081079

§ 371 Date: May 13, 2020

Related U.S. Application Data
(60) Provisional application No. 62/585,682, filed on Nov. 14, 2017.
(60) Provisional application No. 62/719,289, filed on Aug. 17, 2018.

(51) Int. Cl. A61K 47/68; C07D 519/00

(52) U.S. Cl. CPC A61K 47/6803; C07D 519/00; A61K 45/06; A61K 47/6851; A61K 47/6849

(57) ABSTRACT

A conjugate of formula I, wherein Ab is a modified antibody having at least one free conjugation site on each heavy chain.

The present invention relates to conjugates comprising pyrrolobenzodiazepines and related dimers (PBDs), and the precursor drug linkers used to make such conjugates.

### BACKGROUND TO THE INVENTION

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994)). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

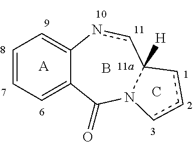

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDeventer, Acc. Chem. Res., 19, 230-237 (1986)). Their ability to form an adduct in the minor groove, enables them to interfere with DNA processing, hence their use as antitumour agents.

It has been previously disclosed that the biological activity of this molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity.

One example of a PBD dimer is SG2000 (SJG-136):

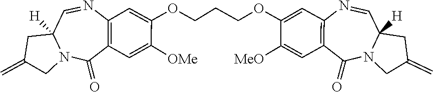

(Gregson, S., et al., J. Med. Chem., 44, 737-748 (2001); Alley, M. C., et al., Cancer Research, 64, 6700-6706 (2004); Hartley, J. A., et al., Cancer Research, 64, 6693-6699 (2004)) which has been involved in clinical trials as a standalone agent, for example, NCT02034227 investigating its use in treating Acute Myeloid Leukemia and Chronic Lymphocytic Leukemia (see: https://www.clinicaltrials.gov/ct2/show/NCT02034227).

Dimeric PBD compounds bearing C2 aryl substituents, such as SG2202 (ZC-207), are disclosed in WO 2005/085251:

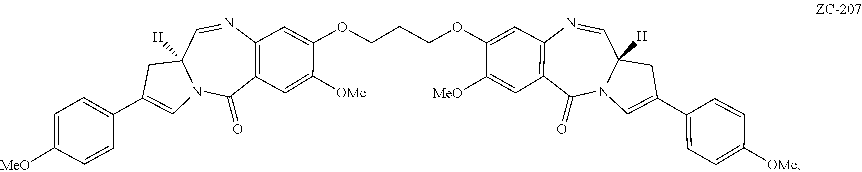 [SMILES: COc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

and in WO2006/111759, bisulphites of such PBD compounds, for example SG2285 (ZC-423):

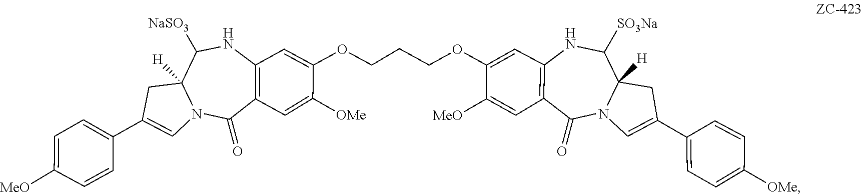

These compounds have been shown to be highly useful cytotoxic agents (Howard, P. W., et al., Bioorg. Med. Chem. (2009), doi: 10.1016/j.bmcl.2009.09.012).

WO 2007/085930 describes the preparation of dimer PBD compounds having linker groups for connection to a cell binding agent, such as an antibody. The linker is present in the bridge linking the monomer PBD units of the dimer.

Dimer PBD compounds having linker groups for connection to a cell binding agent, such as an antibody, are described in WO 2011/130598. The linker in these compounds is attached to one of the available N10 positions, and are generally cleaved by action of an enzyme on the linker group. If the non-bound N10 position is protected with a capping group, the capping groups exemplified have the same cleavage trigger as the linker to the antibody.

Dimer PBD compounds having linker groups for connection to a cell binding agent, such as an antibody, are also described in WO 2011/130613, WO 2011/130616, WO 2013/053873, WO 2013/053871, WO 2013/041606, WO 2013/055993, WO2013/055990, WO 2014/057073 and WO 2015/052321. The linker in these compounds is attached to the PBD core via the C2 position, and are generally cleaved by action of an enzyme on the linker group.

Of particular interest is talirine which when conjugates to an anti-CD33 anitbody forms Vadastuximab talirine (SGN-CD33A)(Sutherland, M, et al., Blood 2013 122:1455-1463; doi: 10.1182/blood-2013-03-491506). This ADC is undergoing clinical trials for the treatment of AML. SGN-CD33A has the general structure:

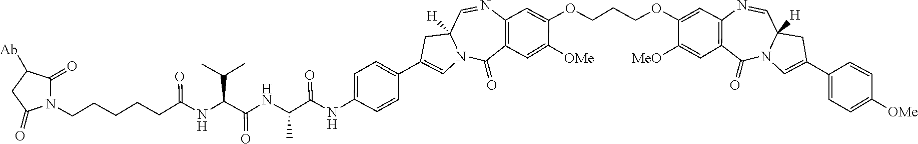

### DISCLOSURE OF THE INVENTION

The present invention provides PBDs, and related PBD dimer conjugates wherein the PBDs are conjugated to antibodies that are modified so as to have at least one free conjugation site on each heavy chain, and where the conjugation is via substituents on both C2 groups of the PBD via respective linkers.

The present invention also provides PBD and related dimer drug linkers, suitable for conjugating to a modified antibodies, where the dimer has a linking groups on substituents on both C2 groups.

A first aspect of the present invention provides a conjugate of formula I:

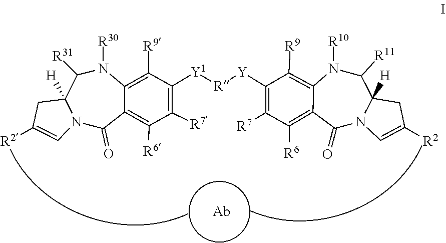

Wherein

Ab is a modified antibody having at least one free conjugation site on each heavy chain; R² is of formula IIIa, formula IIIb or formula IIIc:

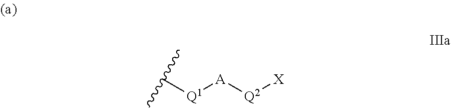

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

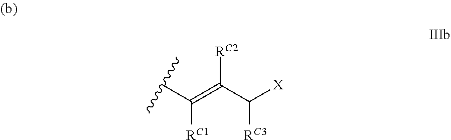

where;

R^{C1}, R^{C2} and R^{C3} are independently selected from H and unsubstituted C₁₋₂ alkyl;

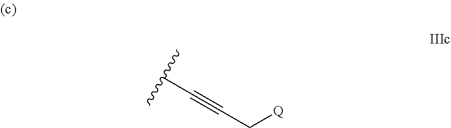

where Q is selected from O—R^{L1}, S—R^{L1} and NR^{N}—R^{L1}, and R^{N} is selected from H, methyl and ethyl

X is selected from the group comprising: O—R^{L1}, S—R^{L1}, CO₂—R^{L1}, CO—R^{L1}, NH—C(═O)—R^{L1}, NHNH—R^{L1}, CONHNH—R^{L1},

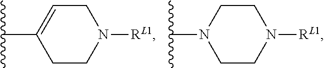

NR^{N}R^{L1}, wherein R^{N} is selected from the group comprising H and 014 alkyl;

R^{L1} is a linker for connection to the antibody (Ab);

R^{2′} is selected from the same groups as R² and is linked to the same antibody;

R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M;

R^{6′}, R^{7′} and R^{9′} are selected from the same groups as R⁶, R⁷ and R⁹ respectively.

It is thought that such ADCs which effectively have a drug antibody ratio (DAR) of 1 could offer significant advantages including reduced off-target toxicity and an enhanced therapeutic window by reducing the minimal effective dose requirement over ADCs consisting of heterogeneous mixtures with higher DARs.

A second aspect of the present invention comprises a compound with the formula II:

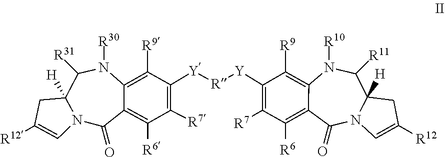

and salts and solvates thereof,

wherein R⁶, R⁷, R⁹, R¹⁰, R¹¹, Y, R″, Y′, R^{6′}, R^{7′}, R^{9′}, R³⁰ and R³¹ are as defined in the first aspect of the invention;

R¹² is of formula IVa, formula IVb or formula IVc:

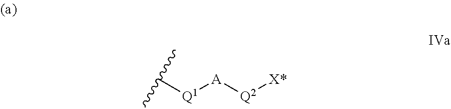

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

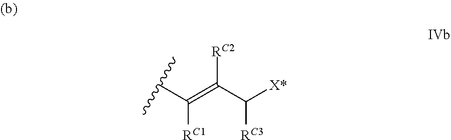

where;

R^{C1}, R^{C2} and R^{C3} are independently selected from H and unsubstituted C₁₋₂ alkyl;

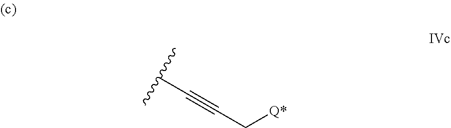

where Q* is selected from O—R^{G1}, S—R^{G1} and NR^{N}—R^{G1}, and R^{N} is selected from H, methyl and ethyl

X* is selected from the group comprising: O—R^{G1}, S—R^{G1}, CO₂—R^{G1}, CO—R^{G1}, NH—C(═O)—R^{G1}, NHNH—R^{G1}, CONHNH—R^{G1},

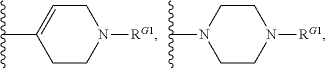

NR^{N}R^{G1}, wherein R^{N} is selected from the group comprising H and C₁₋₄ alkyl;

R^{G1} is a linker for connection to an antibody;

R^{12′} is selected from the same groups as R¹².

A third aspect of the present invention provides the use of a conjugate of the first aspect of the invention in the manufacture of a medicament for treating a proliferative disease. The third aspect also provides a conjugate of the first aspect of the invention for use in the treatment of a proliferative disease. The third aspect also provides a method of treating a proliferative disease comprising administering a therapeutically effective amount of a conjugate of the first aspect of the invention to a patient in need thereof.

One of ordinary skill in the art is readily able to determine whether or not a candidate conjugate treats a proliferative condition for any particular cell type. For example, assays which may conveniently be used to assess the activity offered by a particular compound are described in the examples below.

A fourth aspect of the present invention provides the synthesis of a conjugate of the first aspect of the invention comprising conjugating a compound (drug linker) of the second aspect of the invention with an antibody as defined in the first aspect of the invention.

### BRIEF DESCRIPTION OF FIGURES

FIG. 1 shows schematic representations of (A) modified antibodies suitable for use in the present invention, (B) an antibody drug conjugate comprising a PBD of the present invention;

FIG. 2 shows the heavy and light chain sequences of a Herceptin-Flexmab.

### DEFINITIONS

### Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and

saturated polycyclic hydrocarbon compounds:

norcarane (C₇), norpinane (C₇), norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. The term “C₅₋₇ aryl”, as used herein, pertains to a monovalent moiety obtained by removing a hydrogen atom from an aromatic ring atom of an aromatic compound, which moiety has from 5 to 7 ring atoms and the term “C₅₋₁₀ aryl”, as used herein, pertains to a monovalent moiety obtained by removing a hydrogen atom from an aromatic ring atom of an aromatic compound, which moiety has from 5 to 10 ring atoms. Preferably, each ring has from 5 to 7 ring atoms.

In this context, the prefixes (e.g. C₃₋₂₀, C₅₋₇, C₅₋₆, C₅₋₁₀, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (Si), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Alkoxy: —OR, wherein R is an alkyl group, for example, a C₁₋₇ alkyl group. Examples of C₁₋₇ alkoxy groups include, but are not limited to, —OMe (methoxy),—OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)O(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

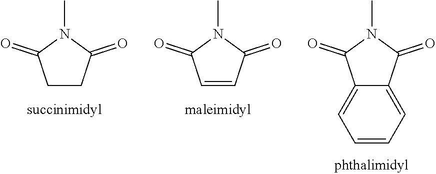

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

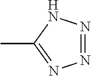

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group.

Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

### Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂-(propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═C H—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

### Ligand Unit

The Ligand Units for use in the present invention are Cell Binding Agents, more specifically modified antibodies, or antigen binding fragments thereof, having at least one conjugation site on each heavy chain. Examples of particular modified antibodies suitable for use according to the present invention are disclosed in WO 2012/064733 (filed as PCT/US2011/059775), which is incorporated herein by reference.

### Antibodies

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include F(ab′)₂, and scFv fragments, and dimeric epitope-binding fragments of any of the above which immunospecifically bind to cancer cell antigens, viral antigens or microbial antigens, single-chain antibody molecules; and multispecific antibodies formed from antibody fragments.

Modified antibodies suitable for use in the present invention include those wherein the native interchain cysteine residues have been substituted for amino acid residues lacking thiol groups. The antibodies may comprise at least one additional substitutions in each heavy chain of an amino acid residue comprising a reactive group suitable for conjugation to a linker. The additionally substituted amino acid may be a cysteine or a non-natural amino acid. The position that is substituted may be selected from those set forth below:

| Antibody | Isotype | IgG1 | IgG2 | IgG3 | IgG4 |
|---|---|---|---|---|---|
| Position | 239 | Ser | Ser | Ser | Ser |
| (Kabat | 282 | Val | Val | Val | Val |
| EU) and | 289 | Thr | Thr | Thr | Thr |
| Corresponding | 297 | Asn | Asn | Asn | Asn |
| Amino Acid | 312 | Asp | Asp | Asp | Asp |
| | 324 | Ser | Ser | Ser | Ser |
| | 330 | Ala | Ala | Ala | Ser |
| | 335 | Thr | Thr | Thr | Thr |
| | 337 | Ser | Ser | Ser | Ser |
| | 339 | Ala | Thr | Thr | Ala |
| | 356 | Glu | Glu | Glu | Glu |
| | 359 | Thr | Thr | Thr | Thr |
| | 361 | Asn | Asn | Asn | Asn |
| | 383 | Ser | Ser | Ser | Ser |
| | 384 | Asn | Asn | Ser | Asn |
| | 398 | Leu | Leu | Leu | Leu |
| | 400 | Ser | Ser | Ser | Ser |
| | 422 | Val | Val | Ile | Val |
| | 440 | Ser | Ser | Ser | Ser |
| | 442 | Ser | Ser | Ser | Ser |

Examples of modified antibodies suitable for use in the present invention include the Flexmab structures disclosed in WO 2012/064733, which is incorporated herein. Such Flexmabs have cysteines with free thiol groups in the hinge region of the antibody that may be used as conjugation sites for linking through the N10 groups of the PBDs of the present invention.

Other examples of modified antibodies suitable for use in the present invention include those where cysteines have been inserted in selected sites in antibodies. These are described in Dimasi, N., et al., Molecular Pharmaceutics, 2017, 14, 1501-1516 (DOI: 10.1021/acs.molpharmaceut.6b00995) and WO2015/157595. In particular, antibodies which have been modified by insertion of a cysteine after the S239 position (ie. between positions 239 and 240) are of use.

Reference is made to the listed on pages 60 to 62 of WO 2012/064733, which is incorporated herein. In some embodiments, the antibody may be to a tumour-associated antigen, for example: HER2 (ErbB2); EPHA2 (EPH receptor A2); CD19; IL2RA (Interleukin 2 receptor, alpha).

Tumour-associate antigens and cognate antibodies for use in embodiments of the present invention are listed below, and are described in more detail on pages 14 to 86 of WO 2017/186894, which is incorporated herein.

(1) BMPR1B (bone morphogenetic protein receptor-type IB)

(2) E16 (LAT1, SLC7A5)

(3) STEAP1 (six transmembrane epithelial antigen of prostate)

(4) 0772P (CA125, MUC16)

(5) MPF (MPF, MSLN, SMR, megakaryocyte potentiating factor, mesothelin)

(6) Napi3b (NAPI-3B, NPTIIb, SLC34A2, solute carrier family 34 (sodium phosphate), member 2, type II sodium-dependent phosphate transporter 3b)

(7) Sema 5b (FLJ10372, KIAA1445, Mm.42015, SEMA5B, SEMAG, Semaphorin 5b Hlog, 25 sema domain, seven thrombospondin repeats (type 1 and type 1-like), transmembrane domain (TM) and short cytoplasmic domain, (semaphorin) 5B)

(8) PSCA hIg (2700050C12Rik, C530008O16Rik, RIKEN cDNA 2700050012, RIKEN cDNA 2700050012 gene)

(9) ETBR (Endothelin type B receptor)

(10) MSG783 (RNF124, hypothetical protein FLJ20315)

(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein)

(12) TrpM4 (BR22450, FLJ20041, TRPM4, TRPM4B, transient receptor potential cation 5 channel, subfamily M, member 4)

(13) CRIPTO (CR, CR1, CRGF, CRIPTO, TDGF1, teratocarcinoma-derived growth factor)

(14) CD21 (CR2 (Complement receptor 2) or C3DR (C3d/Epstein Barr virus receptor) or Hs.73792)

(15) CD79b (CD79B, CD79β, IGb (immunoglobulin-associated beta), B29)

(16) FcRH2 (IFGP4, IRTA4, SPAP1A (SH2 domain containing phosphatase anchor protein 1a), SPAP1B, SPAP1C)

(17) HER2 (ErbB2)

(18) NCA (CEACAM6)

(19) MDP (DPEP1)

(20) IL20R-alpha (IL20Ra, ZCYTOR7)

(21) Brevican (BCAN, BEHAB)

(22) EphB2R (DRT, ERK, Hek5, EPHT3, Tyro5)

(23) ASLG659 (B7h)

(24) PSCA (Prostate stem cell antigen precursor)

(25) GEDA

(26) BAFF-R (B cell-activating factor receptor, BLyS receptor 3, BR3)

(27) CD22 (B-cell receptor CD22-B isoform, BL-CAM, Lyb-8, Lyb8, SIGLEC-2, FLJ22814)

(27a) CD22 (CD22 molecule)

(28) CD79a (CD79A, CD79alpha), immunoglobulin-associated alpha, a B cell-specific protein that covalently interacts with Ig beta (CD79B) and forms a complex on the surface with Ig M molecules, transduces a signal involved in B-cell differentiation), pI: 4.84, MW: 25028 TM: 2 [P] Gene Chromosome: 19q13.2).

(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pI: 8.54 MW: 41959 TM: 7 [P] Gene Chromosome: 11q23.3, (30) HLA-DOB (Beta subunit of MHC class II molecule (Ia antigen) that binds peptides and 20 presents them to CD4+ T lymphocytes); 273 aa, pI: 6.56, MW: 30820.TM: 1 [P] Gene Chromosome: 6p21.3)

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pI: 7.63, MW: 47206 TM: 1 [P] Gene Chromosome: 17p13.3).

(32) CD72 (B-cell differentiation antigen CD72, Lyb-2); 359 aa, pI: 8.66, MW: 40225, TM: 1 5 [P] Gene Chromosome: 9p13.3).

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pI: 6.20, MW: 74147 TM: 1 [P] Gene Chromosome: 5q12).

(34) FcRH1 (Fc receptor-like protein 1, a putative receptor for the immunoglobulin Fc domain that contains C2 type Ig-like and ITAM domains, may have a role in B-lymphocyte differentiation); 429 aa, pI: 5.28, MW: 46925 TM: 1 [P] Gene Chromosome: 1q21-1q22)

(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pI: 6.88, MW: 106468, TM: 1 [P] Gene Chromosome: 1q21)

(36) TENB2 (TMEFF2, tomoregulin, TPEF, HPP1, TR, putative transmembrane proteoglycan, related to the EGF/heregulin family of growth factors and follistatin); 374 aa)

(37) PSMA—FOLH1 (Folate hydrolase (prostate-specific membrane antigen) 1)

(38) SST (Somatostatin Receptor; note that there are 5 subtypes)

(38.1) SSTR2 (Somatostatin receptor 2)

(38.2) SSTR5 (Somatostatin receptor 5)

(38.3) SSTR1

(38.4) SSTR3

(38.5) SSTR4

### AvB6—Both Subunits (39+40)

(39) ITGAV (Integrin, alpha V)

(40) ITGB6 (Integrin, beta 6)

(41) CEACAM5 (Carcinoembryonic antigen-related cell adhesion molecule 5)

(42) MET (met proto-oncogene; hepatocyte growth factor receptor)

(43) MUC1 (Mucin 1, cell surface associated)

(44) CA9 (Carbonic anhydrase IX)

(45) EGFRvIII (Epidermal growth factor receptor (EGFR), transcript variant 3,

(46) CD33 (CD33 molecule)

(47) CD19 (CD19 molecule)

(48) IL2RA (Interleukin 2 receptor, alpha); NCBI Reference Sequence: NM_000417.2);

(49) AXL (AXL receptor tyrosine kinase)

(50) CD30—TNFRSF8 (Tumor necrosis factor receptor superfamily, member 8)

(51) BCMA (B-cell maturation antigen)—TNFRSF17 (Tumor necrosis factor receptor superfamily, member 17)

(52) CT Ags—CTA (Cancer Testis Antigens)

(53) CD174 (Lewis Y)—FUT3 (fucosyltransferase 3 (galactoside 3(4)-L-fucosyltransferase, Lewis blood group)

(54) CLEC14A (C-type lectin domain family 14, member A; Genbank accession no. NM175060)

(55) GRP78—HSPA5 (heat shock 70 kDa protein 5 (glucose-regulated protein, 78 kDa)

(56) CD70 (CD70 molecule) L08096

(57) Stem Cell specific antigens. For example:
5T4 (see entry (63) below)
CD25 (see entry (48) above)
CD32
LGR5/GPR49
Prominin/CD133

(58) ASG-5

(59) ENPP3 (Ectonucleotide pyrophosphatase/phosphodiesterase 3)

(60) PRR4 (Proline rich 4 (lacrimal))

(61) GCC—GUCY2C (guanylate cyclase 2C (heat stable enterotoxin receptor)

(62) Liv-1—SLC39A6 (Solute carrier family 39 (zinc transporter), member 6)

(63) 5T4, Trophoblast glycoprotein, TPBG—TPBG (trophoblast glycoprotein)

(64) CD56—NCMA1 (Neural cell adhesion molecule 1)

(65) CanAg (Tumor associated antigen CA242)

(66) FOLR1 (Folate Receptor 1)

(67) GPNMB (Glycoprotein (transmembrane) nmb)

(68) TIM-1—HAVCR1 (Hepatitis A virus cellular receptor 1)

(69) RG-1/Prostate tumor target Mindin—Mindin/RG-1

(70) B7-H4—VTCN1 (V-set domain containing T cell activation inhibitor 1

(71) PTK7 (PTK7 protein tyrosine kinase 7)

(72) CD37 (CD37 molecule)

(73) CD138—SDC1 (syndecan 1)

(74) CD74 (CD74 molecule, major histocompatibility complex, class II invariant chain)

(75) Claudins—CLs (Claudins)

(76) EGFR (Epidermal growth factor receptor)

(77) Her3 (ErbB3)—ERBB3 (v-erb-b2 erythroblastic leukemia viral oncogene homolog 3 (avian))

(78) RON—MST1R (macrophage stimulating 1 receptor (c-met-related tyrosine kinase))

(79) EPHA2 (EPH receptor A2)

(80) CD20—MS4A1 (membrane-spanning 4-domains, subfamily A, member 1)

(81) Tenascin C—TNC (Tenascin C)

(82) FAP (Fibroblast activation protein, alpha)

(83) DKK-1 (Dickkopf 1 homolog (Xenopus laevis)

(84) CD52 (CD52 molecule)

(85) CS1—SLAMF7 (SLAM family member 7)

(86) Endoglin—ENG (Endoglin)

(87) Annexin A1—ANXA1 (Annexin A1)

(88) V-CAM (CD106)—VCAM1 (Vascular cell adhesion molecule 1)

### Connection of Linker Unit to Ligand Unit

The Ligand unit may be connected to the Linker unit through a disulfide bond.

In one embodiment, the connection between the Ligand unit and the Drug Linker is formed between a thiol group of a cysteine residue of the Ligand unit and a maleimide group of the Drug Linker unit. Other possible groups for linking, and the resulting linking groups, are shown below.

The cysteine residues of the Ligand unit may be available for reaction with the functional group of the Linker unit to form a connection. In other embodiments, for example where the Ligand unit is an antibody, the thiol groups of the antibody may participate in interchain disulfide bonds. These interchain bonds may be converted to free thiol groups by e.g. treatment of the antibody with DTT prior to reaction with the functional group of the Linker unit.

In some embodiments, the cysteine residue is an introduced into the heavy or light chain of an antibody. Positions for cysteine insertion by substitution in antibody heavy or light chains include those described in Published U.S. Application No. 2007-0092940 and International Patent Publication WO2008/070593, which are incorporated herein.

### Methods of Treatment

The compounds of the present invention may be used in a method of therapy. Also provided is a method of treatment, comprising administering to a subject in need of treatment a therapeutically-effective amount of a conjugate of formula I. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate of formula I, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The Conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmune disorder is a T cell-mediated immunological disorder.

In some embodiments, the amount of the Conjugate administered ranges from about 0.01 to about 10 mg/kg per dose. In some embodiments, the amount of the Conjugate administered ranges from about 0.01 to about 5 mg/kg per dose. In some embodiments, the amount of the Conjugate administered ranges from about 0.05 to about 5 mg/kg per dose. In some embodiments, the amount of the Conjugate administered ranges from about 0.1 to about 5 mg/kg per dose. In some embodiments, the amount of the Conjugate administered ranges from about 0.1 to about 4 mg/kg per dose. In some embodiments, the amount of the Conjugate administered ranges from about 0.05 to about 3 mg/kg per dose. In some embodiments, the amount of the Conjugate administered ranges from about 0.1 to about 3 mg/kg per dose. In some embodiments, the amount of the Conjugate administered ranges from about 0.1 to about 2 mg/kg per dose.

### Drug Loading

The drug loading (p) is the average number of PBD drugs per cell binding agent, e.g. antibody. In the present invention, this is always 1. However, any composition may comprise antibodies where a PBD is conjugated and antibodies where a PBD is not conjugated. Thus for a composition, the drug loading (or DAR) may be less than 1, for example 0.75 and higher, 0.80 and higher, 0.85 and higher, 0.90 and higher or 0.95 or higher.

### General Synthetic Routes

The synthesis of PBD compounds is extensively discussed in the following references, which discussions are incorporated herein by reference:

a) WO 00/12508 (pages 14 to 30);

b) WO 2005/023814 (pages 3 to 10);

c) WO 2004/043963 (pages 28 to 29); and

d) WO 2005/085251 (pages 30 to 39).

### Synthesis Route

Compounds of the present invention of formula II:

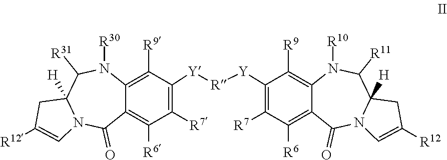

can be synthesised by constructing the C2 linking groups in a manner analogous to that disclosed in WO2010/043880, WO2011/130613, WO2011/130616 and WO2013/041606. Where the C2 linking groups are the same, they can be synthesised in parallel. Where the C2 linking groups are different, orthogonal protection can be used in a similar to in the previous references to synthesise the groups sequentially.

### Synthesis of Drug Conjugates

Antibodies can be conjugated to the Drug Linker compound generally as described in Doronina et al., Nature Biotechnology, 2003, 21, 778-784). Briefly, antibodies (4-5 mg/mL) in PBS containing 50 mM sodium borate at pH 7.4 are reduced with tris(carboxyethyl)phosphine hydrochloride (TCEP) at 37° C. The progress of the reaction, which reduces interchain disulfides, is monitored by reaction with 5,5′-dithiobis(2-nitrobenzoic acid) and allowed to proceed until the desired level of thiols/mAb is achieved. The reduced antibody is then cooled to 0° C. and alkylated with 3 equivalents of drug-linker per antibody. After 1 hour, the reaction is quenched by the addition of 5 equivalents of N-acetyl cysteine. Quenched drug-linker is removed by gel filtration over a PD-10 column. The ADC is then sterile-filtered through a 0.22 μm syringe filter. Protein concentration can be determined by spectral analysis at 280 nm and 329 nm, respectively, with correction for the contribution of drug absorbance at 280 nm. Size exclusion chromatography can be used to determine the extent of antibody aggregation, and RP-HPLC can be used to determine the levels of remaining NAC-quenched drug-linker.

### Further Preferences

The following preferences may apply to all aspects of the invention as described above, or may relate to a single aspect. The preferences may be combined together in any combination.

R^{6′}, R^{7′}, R^{9′} and Y′ are selected from the same groups as R⁶, R⁷, R⁹ and Y respectively. In some embodiments, R^{6′}, R^{7′}, R^{9′} and Y′ are the same as R⁶, R⁷, R⁹ and Y respectively.

### Dimer Link

In some embodiments, Y and Y′ are both O.

In some embodiments, R″ is a C₃₋₇ alkylene group with no substituents. In some of these embodiments, R″ is a C₃, C₅ or C₇ alkylene. In particular, R″ may be a C₃ or C₅ alkylene.

In other embodiments, R″ is a group of formula:

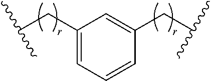

where r is 1 or 2.

R⁶ to R⁹

In some embodiments, R⁹ is H.

In some embodiments, R⁶ is selected from H, OH, OR, SH, NH₂, nitro and halo, and may be selected from H or halo. In some of these embodiments R⁶ is H.

In some embodiments, R⁷ is selected from H, OH, OR, SH, SR, NH₂, NHR, NRR′, and halo. In some of these embodiments R⁷ is selected from H, OH and OR, where R is selected from optionally substituted C₁₋₇ alkyl, C₃₋₁₀ heterocyclyl and C₅₋₁₀ aryl groups. R may be more preferably a C₁₋₄ alkyl group, which may or may not be substituted. A substituent of interest is a C₅₋₆ aryl group (e.g. phenyl). Particularly preferred substituents at the 7-positions are OMe and OCH₂Ph. Other substituents of particular interest are dimethylamino (i.e. NMe₂); —(OC₂H₄)_{q}OMe, where q is from 0 to 2; nitrogen-containing C₆ heterocyclyls, including morpholino, piperidinyl and N-methyl-piperazinyl.

These embodiments and preferences apply to R^{9′}, R^{6′} and R^{7′} respectively.

R¹⁰ and R¹¹

In some embodiments, R¹⁰ and R¹¹ together form a double bond between the nitrogen and carbon atoms to which they are bound.

In some embodiments, R¹¹ is OH.

In some embodiments, R¹¹ is OMe.

In some embodiments, R¹¹ is SO_{z}M, where z is 2 or 3 and M is a monovalent pharmaceutically acceptable cation.

R³⁰ and R³¹

In some embodiments, R³⁰ and R³¹ together form a double bond between the nitrogen and carbon atoms to which they are bound.

In some embodiments, R³¹ is OH.

In some embodiments, R³¹ is OMe.

In some embodiments, R³¹ is SO_{z}M, where z is 2 or 3 and M is a monovalent pharmaceutically acceptable cation.

R¹⁰, R¹¹, R³⁰ and R³¹

In some embodiments, R³⁰ and R³¹ are the same as R¹⁰ and R¹¹ respectively.

R²/R¹²

In some embodiments, R² is of formula IIIa.

A in R² when it is of formula IIIa may be phenyl group or a C₅₋₇ heteroaryl group, for example furanyl, thiophenyl and pyridyl. In some embodiments, A is preferably phenyl.

Q²-X may be on any of the available ring atoms of the C₅₋₇ aryl group, but is preferably on a ring atom that is not adjacent the bond to the remainder of the compound, i.e. it is preferably β or γ to the bond to the remainder of the compound. Therefore, where the C₅₋₇ aryl group (A) is phenyl, the substituent (Q²-X) is preferably in the meta- or para-positions, and more preferably is in the para-position.

In some embodiments, Q¹ is a single bond. In these embodiments, Q² is selected from a single bond and —Z—(CH₂)ₙ—, where Z is selected from a single bond, O, S and NH and is from 1 to 3. In some of these embodiments, Q² is a single bond. In other embodiments, Q² is —Z—(CH₂)ₙ—. In these embodiments, Z may be O or S and n may be 1 or n may be 2. In other of these embodiments, Z may be a single bond and n may be 1.

In other embodiments, Q¹ is —CH═CH—.

In other embodiments, R² is of formula IIIb. In these embodiments, R^{C1}, R^{C2} and R^{C3} are independently selected from H and unsubstituted C₁₋₂ alkyl. In some preferred methyl. In certain embodiments, R^{C1}, R^{C2} and R^{C3} are independently selected from H and methyl.

X is a group selected from the list comprising: O—R^{L1}, S—R^{L1}, CO₂—R^{L1}, CO—R^{L1}, NH—C(═O)—R^{L1}, NHNH—R^{L1}, CONHNH—R^{L1},

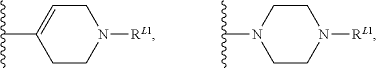

NR^{N}R^{L1}, wherein R^{N} is selected from the group comprising H and C₁₋₄ alkyl. X may preferably be: O—R^{L1}, S—R^{L1}, CO₂—R^{L1}, NH—C(═O)—R^{L1} or NR^{N}R^{L1}. Particularly preferred groups include: O—R^{L1}, S—R^{L1}, and NH—R^{L1}, with NH—R^{L1} being the most preferred group.

In some embodiments R² is of formula IIIc. In these embodiments, it is preferred that Q is NR^{N}—R^{L1}. In other embodiments, Q is O—R^{L1}. In further embodiments, Q is S—R^{L1}. R^{N} is preferably selected from H and methyl. In some embodiment, R^{N} is H. In other embodiments, R^{N} is methyl.

In some embodiments, R² may be -A-CH₂—X and -A-X. In these embodiments, X may be O—R^{L1}, S—R^{L1}, CO₂—R^{L1}, CO—R^{L1} and NH—R^{L1}. In particularly preferred embodiments, X may be NH—R^{L1}.

The above preferences apply to R¹² as appropriate (with R^{L1} being replaced by R^{G1}). The above preferences also apply to R^{2′} and R^{12′}.

These embodiments and preferences also apply to the second aspect of the invention.

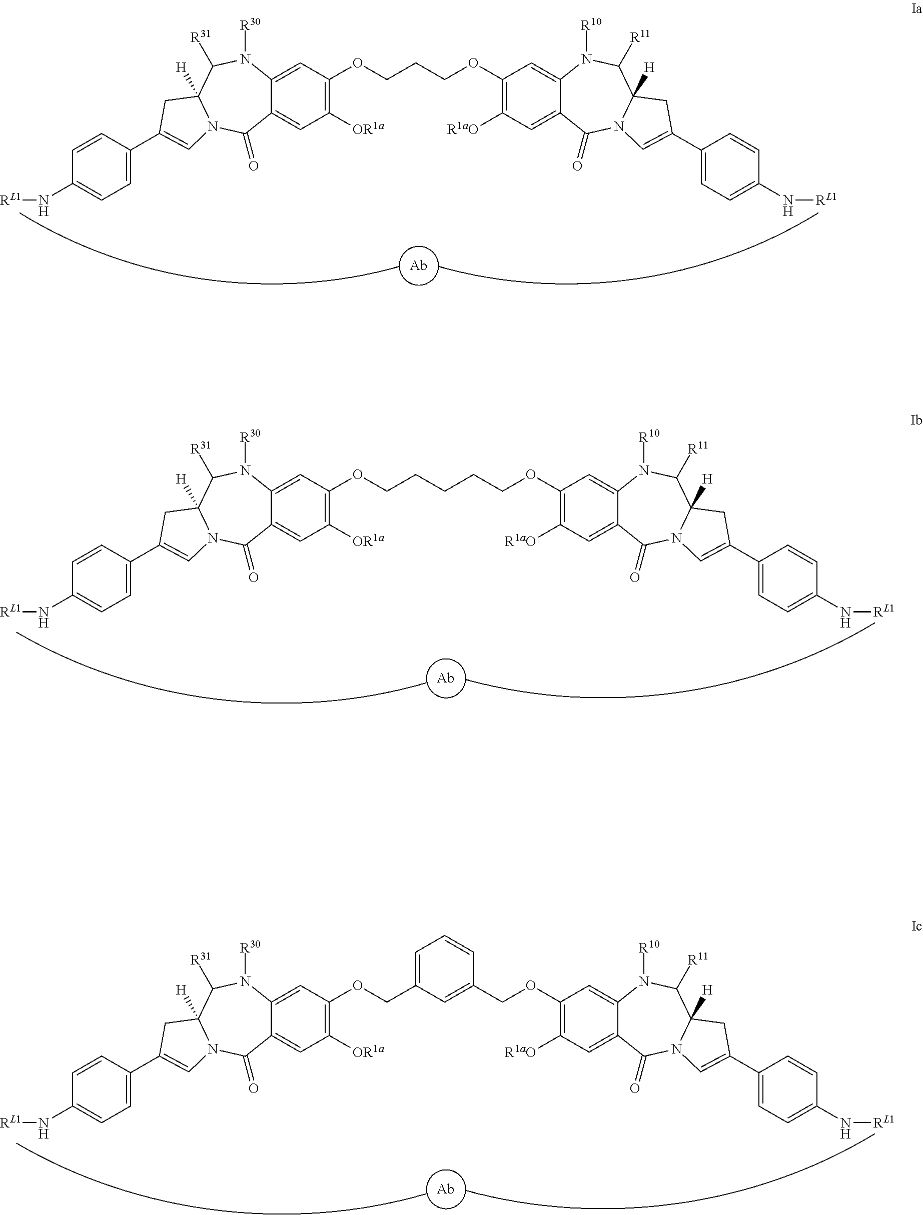

where

R^{1a} is selected from methyl and benzyl;

R^{L1}, R¹⁰, R¹¹, R³⁰ and R³¹ are as defined elsewhere.

Linker (R^{L1} and R^{G1})

In some embodiments, R^{L1} is Q^{X}-Z-G^{LL}.

In some embodiments, R^{G1} is Q^{X}-Z-G^{L}.

### Q^{X}

In one embodiment, Q^{X} is an amino acid residue. The amino acid may a natural amino acids or a non-natural amino acid.

In one embodiment, Q^{X} is selected from: Phe, Lys, Val, Ala, Cit, Leu, Ile, Arg, and Trp, where Cit is citrulline.

In one embodiment, Q^{X} comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q^{X} is selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{CH},
^{CO}-Phe-Cit-^{NH},
^{CO}-Leu-Cit-^{NH},
^{CO}-Ile-Cit-^{NH},
^{CO}-Phe-Arg-^{NH}, and
^{CO}-Trp-Cit-^{NH};

where Cit is citrulline.

Preferably, Q^{X} is selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{NH}.

Most preferably, Q^{X} is selected from ^{CO}-Phe-Lys-^{NH}, ^{CO}-Val-Cit-^{NH} and ^{CO}-Val-Ala-^{NH}.

Other dipeptide combinations of interest include:
^{CO}-Gly-Gly-^{NH},
^{CO}-Pro-Pro-^{NH}, and
^{CO}-Val-Glu-^{NH}.

Other dipeptide combinations may be used, including those described by Dubowchik et al., Bioconjugate Chemistry, 2002, 13, 855-869, which is incorporated herein by reference.

In some embodiments, Q^{X} is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin.

In one embodiment, the amino acid side chain is chemically protected, where appropriate. The side chain protecting group may be a group as discussed below. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

### Z

Z is:

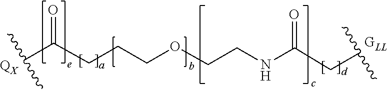

where a=0 to 5, b=0 to 16, c=0 or 1, d=0 to 5 and e is 0 or 1.

a may be 0, 1, 2, 3, 4 or 5. In some embodiments, a is 0 to 3. In some of these embodiments, a is 0 or 1. In further embodiments, a is 0.

b may be 0, 1, 2, 3, 4, 5, 6, 7, 8, 9, 10, 11, 12, 13, 14, 15 or 16. In some embodiments, b is 0 to 12. In some of these embodiments, b is 0 to 8, and may be 0, 2, 4 or 8.

c may be 0 or 1.

d may be 0, 1, 2, 3, 4 or 5. In some embodiments, d is 0 to 3. In some of these embodiments, d is 1 or 2. In further embodiments, d is 2.

e may be 0 or 1.

In some embodiments of Z, a is 0, b is 0, c is 0, d is 5 and e is 1.

In some embodiments of Z, a is 0, c is 1, d is 2, e is 1, and b may be from 0 to 8. In some of these embodiments, b is 0, 4 or 8.

### G^{LL}

G^{LL} may be selected from:

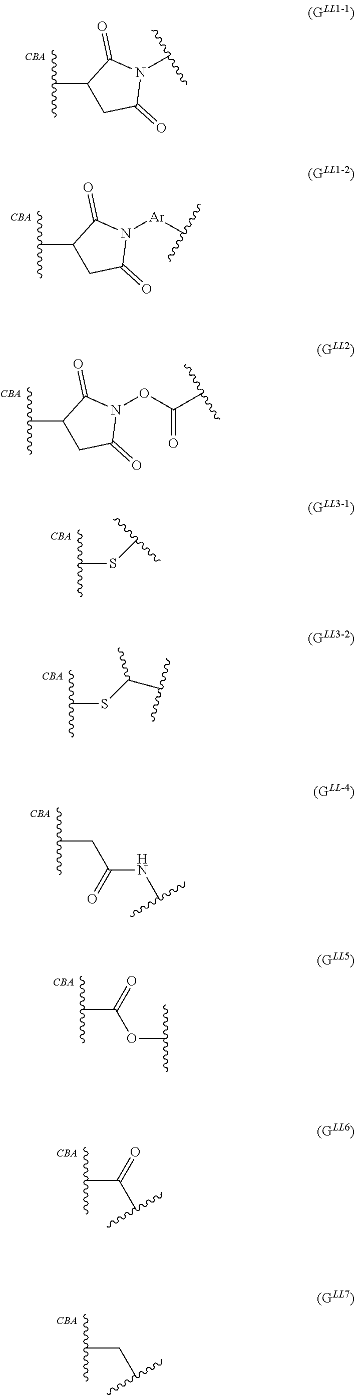

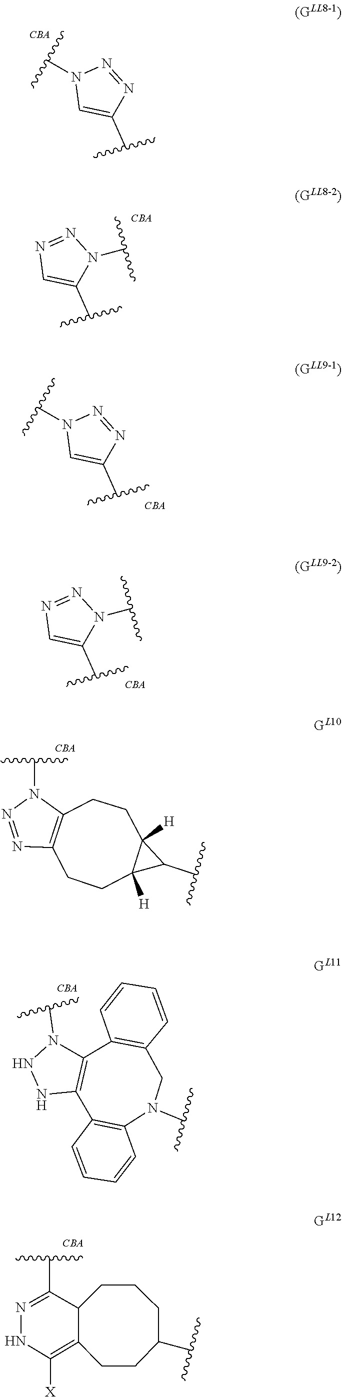

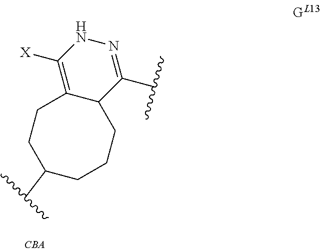

where Ar represents a C₅₋₆ arylene group, e.g. phenylene and X represents C¹⁻⁴ alkyl.

In some embodiments, G^{LL} is selected from G^{LL1-1} and G^{LL1-2}. In some of these embodiments, G^{LL} is G^{LL1-1}.

### G^{L}

G^{L} may be selected from

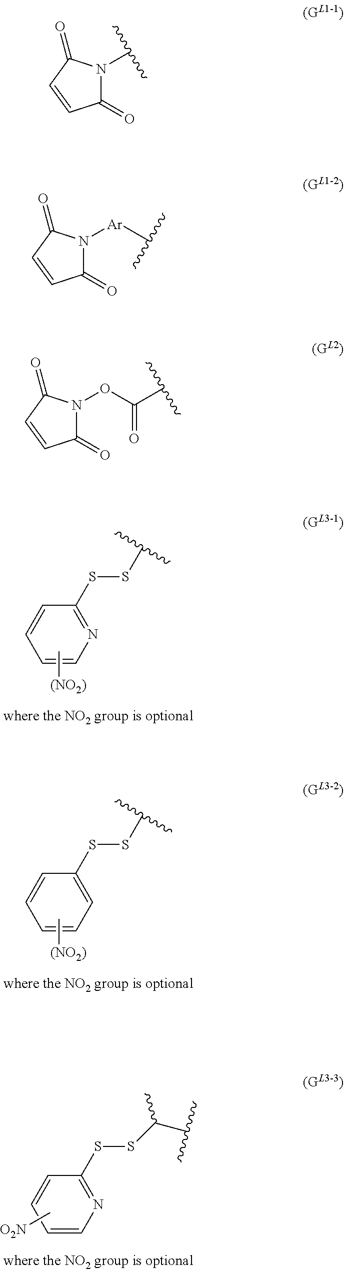

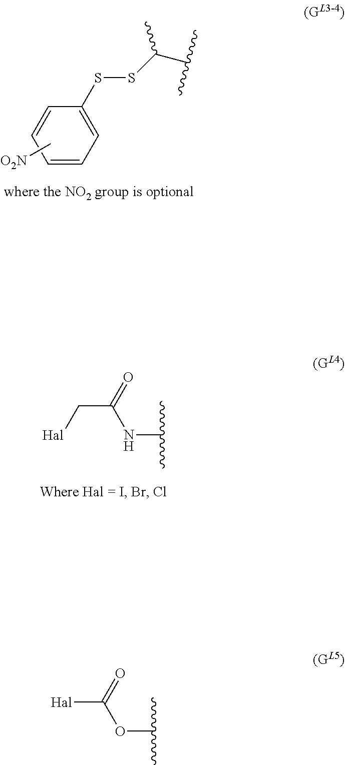

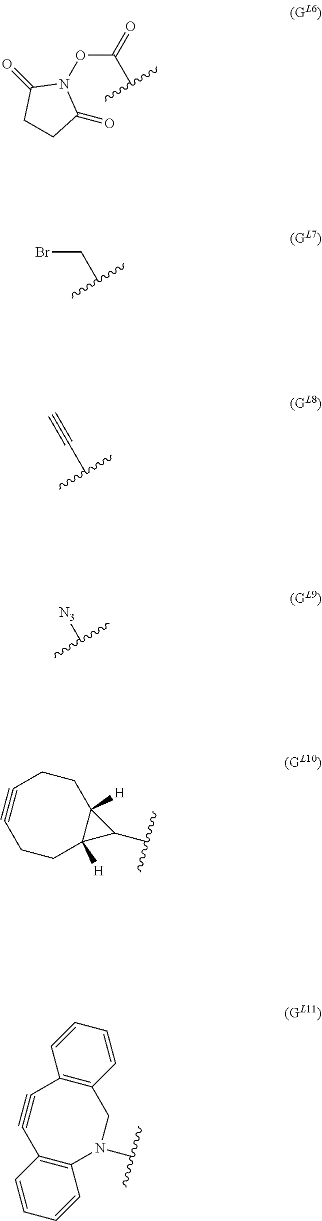

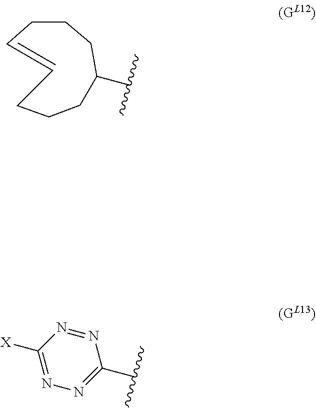

where Ar represents a C₅₋₆ arylene group, e.g. phenylene, and X represents C¹⁻⁴ alkyl

In some embodiments, G^{L} is selected from G^{L1-1} and G^{L1-2}. In some of these embodiments, G^{L} is G^{L1-1}.

In one particular embodiment, the first aspect of the invention comprises a conjugate of formula Id:

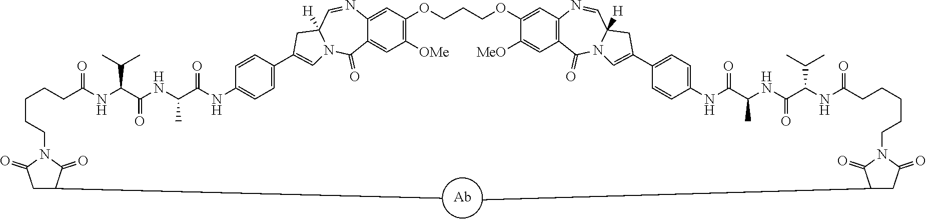 [SMILES: COc1cc2c(cc1OCCCOc1cc3c(cc1OC)C(=O)N1C=C(c4ccc(NC(=O)[C@H](C)NC(=O)[C@@H](NC(=O)CCCCCN5C(=O)CC(C)C5=O)C(C)C)cc4)C[C@H]1C=N3)N=C[C@@H]1CC(c3ccc(NC(=O)[C@H](C)NC(=O)[C@@H](NC(=O)CCCCCN4C(=O)CC(C)C4=O)C(C)C)cc3)=CN1C2=O]

In one particular embodiment, the first aspect of the invention comprises a conjugate of formula Ie:

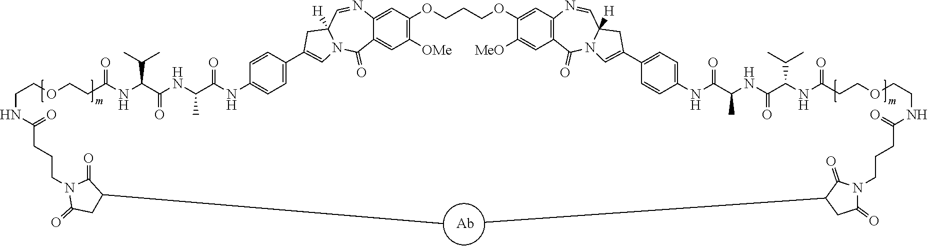

where m is an integer from 2 to 8.

In one particular embodiment, the second aspect of the invention, the Drug linker (D^{L}) is of formula (Id′):

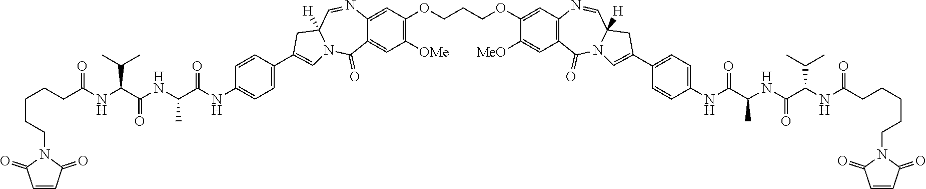 [SMILES: COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(NC(=O)[C@H](C)NC(=O)[C@@H](NC(=O)CCCCCN4C(=O)C=CC4=O)C(C)C)cc3)=CN1C2=O]

In one particular embodiment, the second aspect of the invention, the Drug linker (D^{L}) is of formula (Ie′):

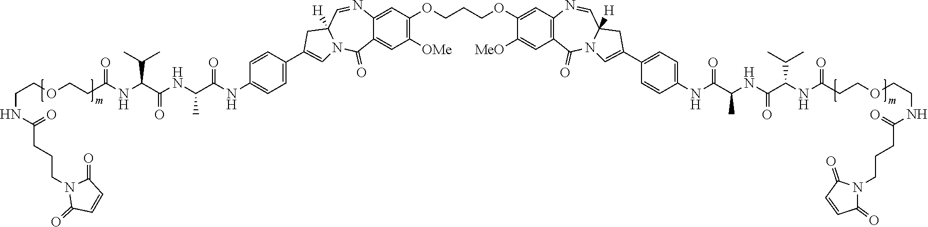

where m is an integer from 2 to 8.

In some embodiments, each R^{L1} are different. In other embodiments, both R^{L1} are the same.

In some embodiments, each R^{G1} are different. In other embodiments, both R^{G1} are the same.

In particular, in embodiments where the linking groups are different, differences may only be in the G groups, such that the remainder of the linking groups are the same (so that the cleavage triggers are the same).

In some embodiments of the present invention, the C11 substituent may be in the following stereochemical arrangement relative to neighbouring groups:

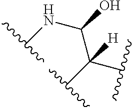

In other embodiments, the C11 substituent may be in the following stereochemical arrangement relative to neighbouring groups:

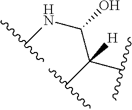

Compounds of particular interest include those of the examples.

### EXAMPLES

Reaction progress was monitored by thin-layer chromatography (TLC) using Merck Kieselgel 60 F254 silica gel, with fluorescent indicator on aluminium plates.

Visualisation of TLC was achieved with UV light or iodine vapour unless otherwise stated. Flash chromatography was performed using Merck Kieselgel 60 F254 silica gel. Extraction and chromatography solvents were bought and used without further purification from Fisher Scientific, U.K. All chemicals were purchased from Aldrich, Lancaster or BDH.

¹H and ¹³C NMR spectra were obtained on a Bruker Avance 400 spectrometer. Coupling constants are quoted in hertz (Hz). Chemical shifts are recorded in parts per million (ppm) downfield from tetramethylsilane. Spin multiplicities are described as s (singlet), bs (broad singlet), d (doublet), t (triplet), q (quartet), p (pentuplet) and m (multiplet).

The LC/MS conditions were as follow:

LCMS data were obtained using a Shimadzu Nexera series LC/MS with a Shimadzu LCMS-2020 quadrupole MS, with Electrospray ionisation. Mobile phase A—0.1% formic acid in water. Mobile phase B—0.1% formic acid in acetonitrile.

LCMS 3 min: initial composition was 5% B held over 0.25 min, then increase from 5% B to 100% B over a 2 min period. The composition was held for 0.50 min at 100% B, then returned to 5% B in 0.05 minutes and hold there for 0.05 min. Total gradient run time equals 3 min.

Flow rate 0.8 mL/min. Wavelength detection range: 190 to 800 nm. Oven temperature: 50° C. Column: Waters Acquity UPLC BEH Shield RP18 1.7 μm 2.1×50 mm.

LCMS 15 min: initial composition 5% B held over 1 min, then increase from 5% B to 100% B over a 9 min period. The composition was held for 2 min at 100% B, then returned to 5% B in 0.10 minutes and hold there for 3 min. Total gradient run time equals 15 min. Flow rate 0.6 mL/min. Wavelength detection range: 190 to 800 nm. Oven temperature: 50° C. Column: ACE Excel 2 C18-AR, 2μ, 3.0×100 mm.

### Example 1

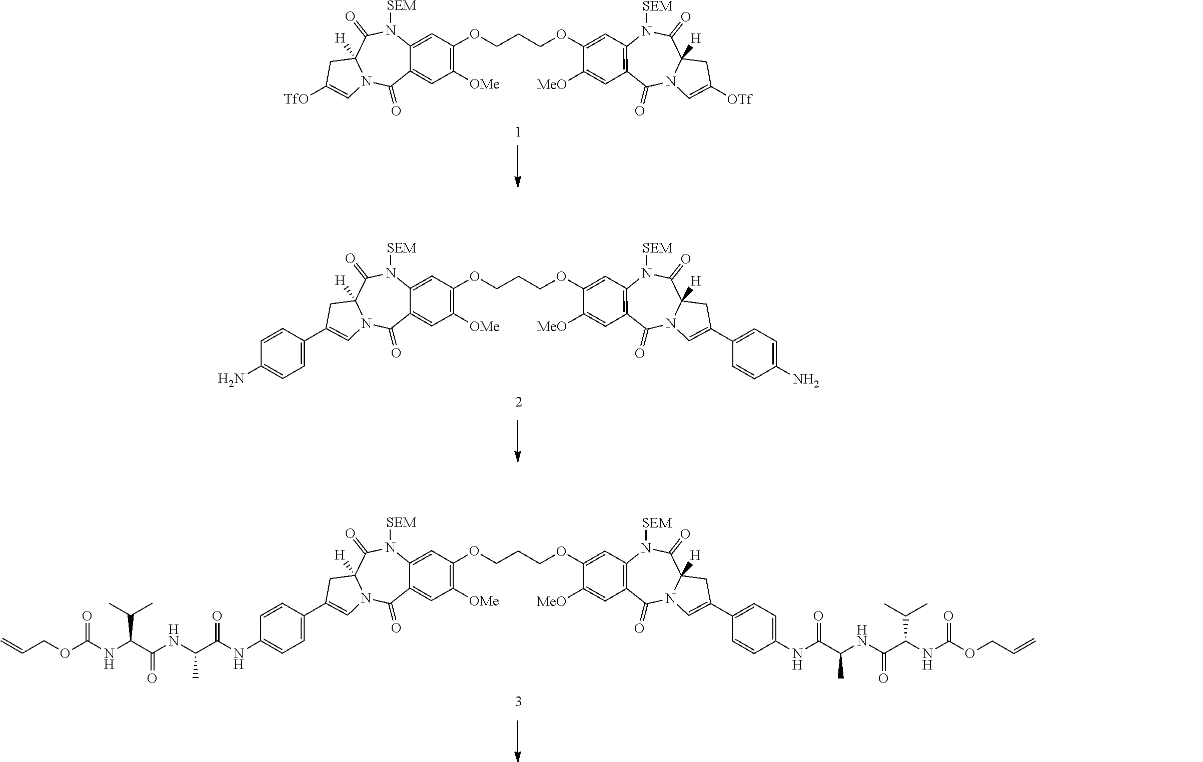

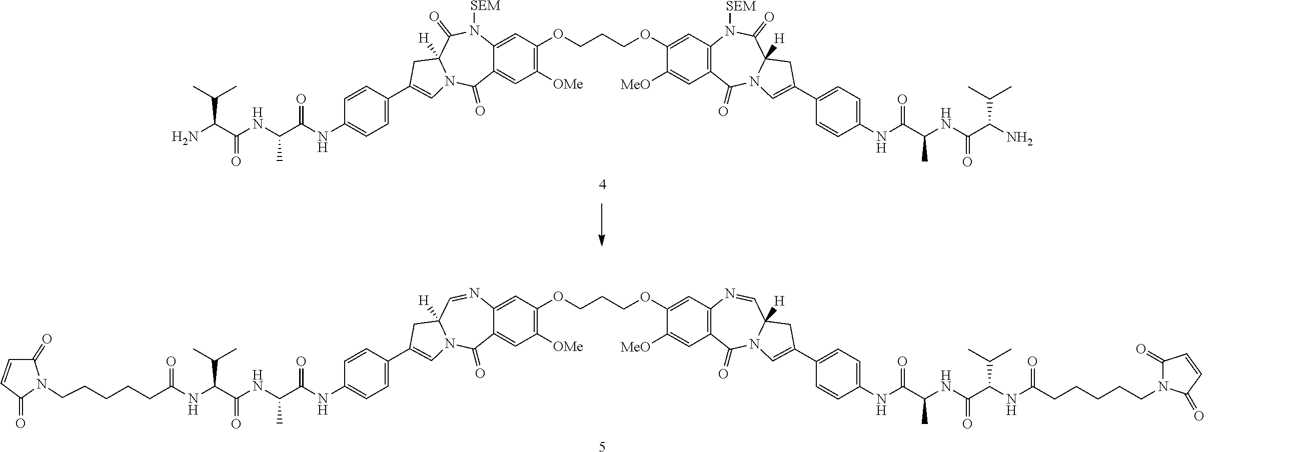 [SMILES: COc1cc2c(cc1OCCCOc1cc3c(cc1OC)C(=O)N1C=C(c4ccc(NC(=O)[C@H](C)NC(=O)[C@@H](N)C(C)C)cc4)C[C@H]1C(=O)N3COCC[Si](C)(C)C)N(COCC[Si](C)(C)C)C(=O)[C@@H]1CC(c3ccc(NC(=O)[C@H](C)NC(=O)[C@@H](N)C(C)C)cc3)=CN1C2=O ; "4" ; COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(NC(=O)[C@H](C)NC(=O)[C@@H](NC(=O)CCCCCN4C(=O)C=CC4=O)C(C)C)cc3)=CN1C2=O ; "5"]

Compound (1) is Compound 8a of WO2010/043880.

### a) (11aS,11a′S)-8,8′-(propane-1,3-diylbis(oxy))bis(2-(4-aminophenyl)-7-methoxy-10-((2-(trimethylsilyl)ethoxy)methyl)-1,11a-dihydro-5H-benzo[e]pyrrolo[1,2-a][1,4]diazepine-5,11(10H)-dione) (2)

Pd(PPh₃)₄ (164 mg, 0.14 mmol) was added to a stirred mixture of the bis-enol triflate 1 (4 g, 3.6 mmol), boronic ester (1.96 g, 8.9 mmol) and Na₂CO₃ (3.41 g, 32.2 mmol) in a 2:1:1 mixture of toluene/MeOH/H₂O (80 mL). The reaction mixture was allowed to stir at 30° C. under a nitrogen atmosphere for 1 h after which time all of bis-enol triflate 1 has reacted. The reaction mixture was then evaporated to dryness before the residue was taken up in CH₂Cl₂ (150 mL) and washed with H₂O (2×75 mL), brine (75 mL), dried (MgSO₄), filtered and evaporated under reduced pressure to provide the crude product. Purification by flash chromatography (gradient elution: 1:1 v/v Hexane/EtOAc to 100% EtOAc) afforded product 2 as a dark orange foam (3.08 g, 86%). LC/MS 1.88 min (ES+) m/z=1003.30 [M+H]⁺.

### b) Diallyl ((2S,2′S)-(((2S,2′S)-((((11aS,11a′S)-(propane-1,3-diylbis(oxy))bis(7-methoxy-5,11-dioxo-10-((2-(trimethylsilyl)ethoxy)methyl)-5,10,11,11a-tetrahydro-1H-benzo[e]pyrrolo[1,2-a][1,4]diazepine-8,2-diyl))bis(4,1-phenylene))bis(azanediyl))bis(1-oxopropane-1,2-diyl))bis(azanediyl))bis(3-methyl-1-oxobutane-1,2-diyl))dicarbamate (3)

To a solution of 2 (2.71 g, 2.7 mmol) in dry CH₂Cl₂ (40 mL) was added the protected peptide (1.61 g, 5.9 mmol) and EEDQ (1.46 mg, 5.9 mmol). The mixture was stirred at room temperature until completion (16h). The reaction mixture was then washed with H₂O (2×50 mL), brine (50 mL), dried (MgSO₄), filtered and evaporated under reduced pressure to provide the crude product. Purification by flash chromatography (gradient elution: 100% CHCl₃ to 93/7 CHCl₃/MeOH) afforded product 3 as a yellow foam. LC/MS 1.90 min (ES+) m/z=1511.65 [M+H]⁺.

### c) (2S,2′S)—N,N′-((2S,2′S)-((((11aS,11a′S)-(propane-1,3-diylbis(oxy))bis(7-methoxy-5,11-dioxo-10-((2-(trimethylsilyl)ethoxy)methyl)-5,10,11,11a-tetrahydro-1H-benzo[e]pyrrolo[1,2-a][1,4]diazepine-8,2-diyl))bis(4,1-phenylene))bis(azanediyl))bis(1-oxopropane-1,2-diyl))bis(2-amino-3-methylbutanamide) (4)

Pyrrolidine (1.1 mL, 13 mmol) and Pd(PPh3)4 (183 mg, 0.16 mmol) were added to a solution of 3 (assumed 100%, 2.69 mmol) in dry CH₂Cl₂ (40 mL). The reaction mixture was diluted with CH₂Cl₂ (60 mL) and the organic phase was washed with H₂O (2×100 mL) and brine (100 mL). The organic phase was dried over MgSO₄, filtered and excess solvent removed by rotary evaporation under reduced pressure. Purification by flash chromatography (gradient elution: 100% CHCl₃ to 93/7 CHCl₃/MeOH) afforded product 4 as a yellow foam (1.5 g, 41% yield. LC/MS 1.33 min (ES+) m/z=1344.40 ([M+H]⁺.

### d) N,N′-((2S,2′S)-(((2S,2′S)-((((11aS,11a′S)-(propane-1,3-diylbis(oxy))bis(7-methoxy-5-oxo-5,11a-dihydro-1H-benzo[e]pyrrolo[1,2-a][1,4]diazepine-8,2-diyl))bis(4,1-phenylene))bis(azanediyl))bis(1-oxopropane-1,2-diyl))bis(azanediyl))bis(3-methyl-1-oxobutane-1,2-diyl))bis(6-(2,5-dioxo-2,5-dihydro-1H-pyrrol-1-yl)hexanamide) (5)

A solution of Super-Hydride® (0.186 mL, 1M in THF) was added dropwise to a solution of SEM-dilactam 4 (100 mg, 0.074 mmol) in dry THF (5 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3×50 mL) and the combined organics were washed with brine (100 mL), dried with MgSO₄, filtered and the solvent removed by rotary evaporation under reduced pressure. The crude product was dissolved in dry CH₂Cl₂ in a round bottom flask purged with argon. Maleimide caproic acid (31.4 mg, 0.148 mmol) and EDCl.HCl (28.5 mg, 0.148 mmol) were added and the mixture was left to stir at room temperature. After a couple of hours, more maleimide caproic acid (5 mg) and EDCl.HCl (5 mg) were added to push the reaction to completion. The crude material was purified by reverse phase HPLC to afford product 5 with 80% purity (2.2 mg, 2% yield). LC/MS 1.46 min (ES+) m/z=1438.25 [M+H]+; LC/MS15 min 6.20 min (ES+) m/z=1438.20 [M+H]⁺.

### Example 2

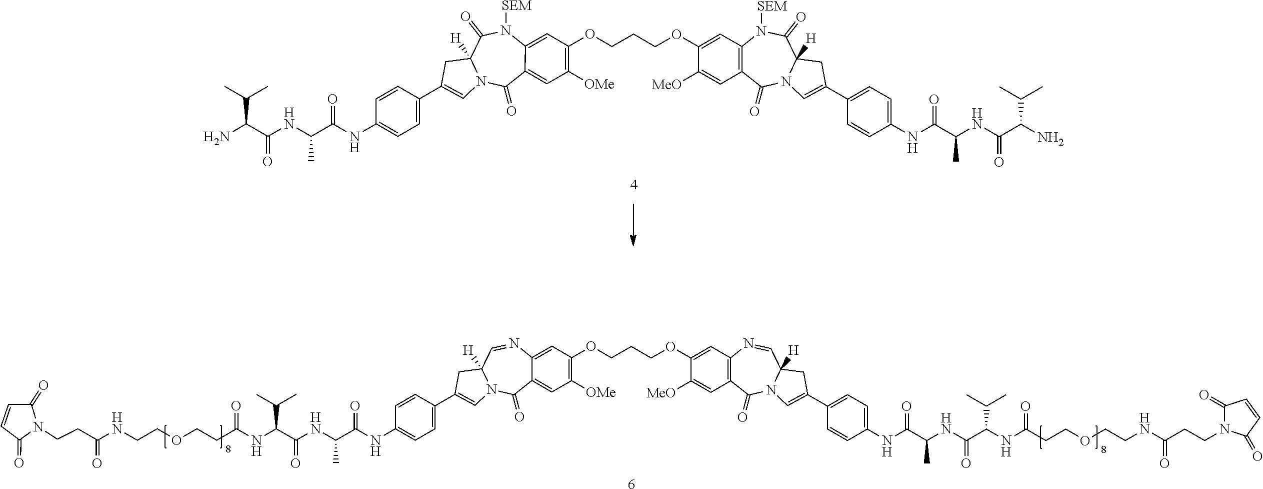

### N,N′-((2S,2′S)-(((2S,2′S)-((((11aS,11a′S)-(propane-1,3-diylbis(oxy))bis(7-methoxy-5-oxo-5,11a-dihydro-1H-benzo[e]pyrrolo[1,2-a][1,4]diazepine-8,2-diyl))bis(4,1-phenylene))bis(azanediyl))bis(1-oxopropane-1,2-diyl))bis(azanediyl))bis(3-methyl-1-oxobutane-1,2-diyl))bis(1-(3-(2,5-dioxo-2,5-dihydro-1H-pyrrol-1-yl)propanamido)-3,6,9,12,15,18,21,24-octaoxaheptacosan-27-amide) (6)

A solution of Super-Hydride® (0.37 mL, 1M in THF) was added dropwise to a solution of SEM-dilactam 4 (200 mg, 0.15 mmol) in dry THF (5 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3×50 mL) and the combined organics were washed with brine (100 mL), dried with MgSO₄, filtered and the solvent removed by rotary evaporation under reduced pressure. The crude product was dissolved in dry CH₂Cl₂ in a round bottom flask purged with argon. Mal-dPEG₈-OH acid (31.4 mg, 0.148 mmol) and EDCl.HCl (28.5 mg, 0.148 mmol) were added and the mixture was left to stir at room temperature until complete. The crude material was purified by reverse phase HPLC to afford product 6 with 82% purity (37 mg, 11% yield). LC/MS 1.38 min (ES+) m/z=1101.05 [M+2H]^{2+.}; LC/MS₁₅ₘᵢₙ 5.84 min (ES+) m/z=1101.05 [M+2H]²⁺.

### Production of Herceptin-Flexmab and NIP228-Flexmab Antibodies

### General

Cell lines SKBR-3 (HER2⁺, 1.5×10⁶ receptors/cell), MDA-MB-453 (HER2⁺, 7.7×10⁴ receptors/cell), and MCF-7 (HER2⁻) were obtained from ATCC and maintained in T175 tissue culture flasks (Corning) using the manufacturer's recommended media (SKBR-3: McCoys 5A+10% FBS, MDA-MB-453: DMEM+10% FBS, and MCF-7: DMEM+10% FBS). 293F cells (Invitrogen) used for transfection were maintained in 293F Freestyle media (Invitrogen). SKBR-3, MDA-MB-453, and MCF-7 cells were cultured in a 37° C. incubator with 5% CO₂. 293F cells were cultured in shake flasks (2 L, Corning) at 37° C. with 8% CO₂ and rotation at 120 rpm. All reagents were purchased from Sigma Aldrich, VWR, or JT Baker unless otherwise specified and used without additional purification.

### Design and Construction of Herceptin-Flexmab and NIP228-Flexmab Antibodies

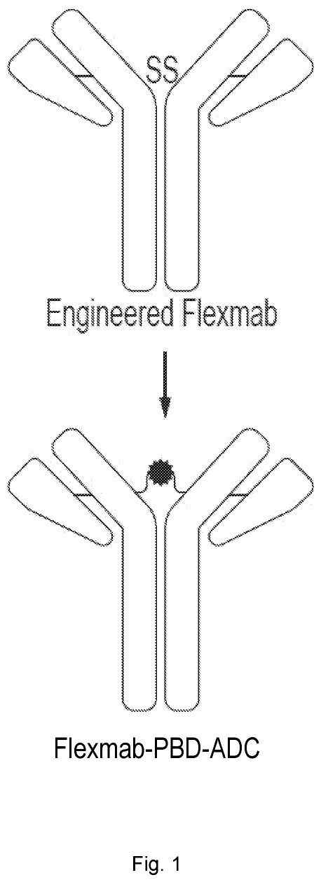

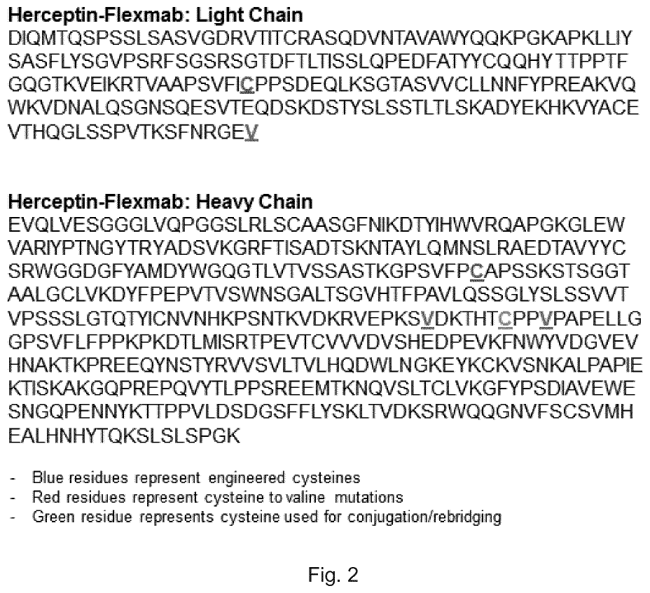

The Herceptin wild-type antibody was used as the template to engineer the Herceptin-Flexmab. The light chain of the Herceptin-Flexmab consists of two mutations, F118C and C214V, whereas the heavy chain contains three mutations, L124C, C216V, and C225V (see FIGS. 1 and 2). The F118C mutation in the light chain forms a disulfide bond with the L124C mutation in the heavy chain. This engineered disulfide is not solvent exposed, but serves to preserve the covalent linkage between the light and heavy chains. The C222 hinge cysteine was left unmodified and served as the location for site-specific conjugation with the pBD-based drug linker. The light chain and heavy chain sequences for the Herceptin-Flexmab were codon-optimized for mammalian expression and procured from GeneArt (Life Technologies). The optimized Herceptin-Flexmab construct was subcloned with standard molecular biology techniques using the BssHII/NheI sites (light chain) and the SalI/NotI sites (heavy chain) into a MedImmune proprietary mammalian expression vector which contains an IgG light chain signal peptide for secretion and cytomegalovirus promoters for recombinant expression. The completed mammalian expression plasmid, pOE-Herceptin-Flexmab was confirmed by DNA sequencing. The negative control NIP228-Flexmab antibody was generated as described for the Herceptin-Flexmab while using the wild-type NIP228 antibody (MedImmune proprietary) as a template.

### Expression and Purification of Herceptin-Flexmab and NIP228-Flexmab Antibodies

Expression and purification of Herceptin-Flexmab and NIP228-Flexmab antibodies was conducted according to previously published methods (Dimasi, N., et al., Journal of Molecular Biology, 2009, 393, 672-692; DOI: 10.1016/j.jmb.2009.08.032). Following transient 293F expression and protein-A purification, the antibodies were formulated into conjugation buffer (1×PBS, 0.1 mM EDTA, pH 7.2) using Slide-A-Lyzer dialysis cassettes at 4° C. (10 kDa MWCO, Thermo) and concentrated to 8.0 mg/mL (Herceptin-Flexmab) and 5.52 mg/mL (NIP228-Flexmab) using Vivaspin concentrators (10 kDa MWCO, GE Healthcare). Final concentrations were determined using a Nanodrop spectrophotometer (A₂₈₀, Thermo). Transient expression yields after 6 days were 500 mg/L and 150 mg/L for Herceptin-Flexmab and NIP228-Flexmab, respectively.

### Conjugations

Herceptin and R347 antibodies engineered to have cysteine inserted between the 239 and 240 positions were produced following the methods described in Dimasi, N., et al., Molecular Pharmaceutics, 2017, 14, 1501-1516 (DOI: 10.1021/acs.molpharmaceut.6b00995).

Conjugations were carried out to the following antibodies with Compound 6: Herceptin-C239i; and R347-C239i with an antibody concentration of 4.0 mg/ml, 6 equivalents of compound 6 and a run time of 1 hour. Formation of DAR=1 species was observed.

All documents and other references mentioned above are herein incorporated by reference.

1.-86. (canceled)

87. A conjugate of formula I: 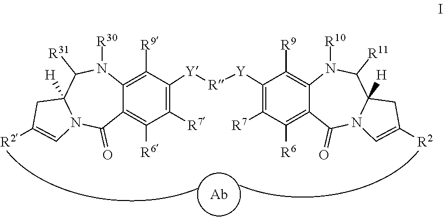
wherein
Ab is a modified antibody having at least one free conjugation site on each heavy chain
R² is of formula IIIa, formula IIIb or formula IIIc: 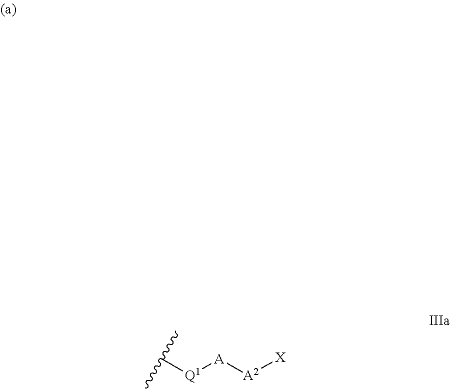
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 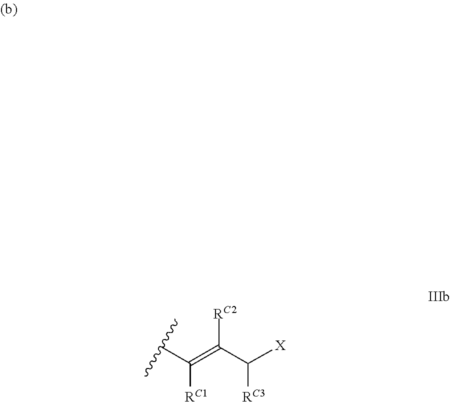
where;
R^{C1}, R^{C2} and R^{C3} are independently selected from H and unsubstituted C₁₋₂ alkyl; 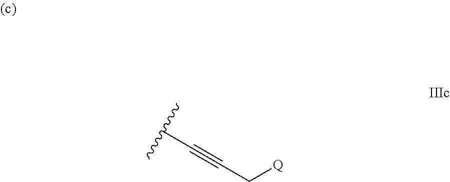
where Q is selected from O—R^{L1}, S—R^{L1} and NR^{N}—R^{L1}, and R^{N} is selected from H, methyl and ethyl
X is selected from the group comprising: O—R^{L1}, S—R^{L1}, CO₂—R^{L1}, CO—R^{L1}, NH—C(═O)—R^{L1}, NHNH—R^{L1}, CONHNH—R^{L1}, 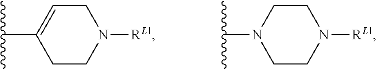
NR^{N}R^{L1}, wherein R^{N} is selected from the group comprising H and C₁₋₄ alkyl;
R^{L1} is a linker for connection to the antibody (Ab);
R^{2′} is selected from the same groups as R² and is linked to the same antibody;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M;
R^{6′}, R^{7′} and R^{9′} are selected from the same groups as R⁶, R⁷ and R⁹ respectively.

88. A conjugate according to claim 87, wherein:
a) both Y and Y′ are O;
b) R″ is C₃₋₇ alkylene;
c) R″ is a group of formula: 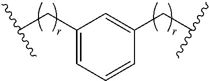
where r is 1 or 2;
d) R⁹ is H;
e) R⁶ is H;
f) R⁷ is selected from H, OH and OR optionally wherein R⁷ is a C₁₋₄ alkyloxy group.

89. A conjugate according to claim 87, wherein:
a) R¹⁰ and R¹¹ together form a double bond between the nitrogen and carbon atoms to which they are bound;
b) R¹⁰ is H and R¹¹ is OH;
c) R¹⁰ is H and R¹¹ is OMe;
d) R¹⁰ is H and R¹¹ is SO_{z}M, where z is 2 or 3 and M is a monovalent pharmaceutically acceptable cation.

90. A conjugate according to claim 87, wherein:
a) R³⁰ and R³¹ together form a double bond between the nitrogen and carbon atoms to which they are bound;
b) R³⁰ is H and R³¹ is OH;
c) R³⁰ is H and R³¹ is OMe;
d) R³⁰ is H and R³¹ is SO_{z}M, where z is 2 or 3 and M is a monovalent pharmaceutically acceptable cation.

91. A conjugate according to claim 87, wherein:
a) R² is of formula IIIa and A is phenyl;
b) R² is of formula IIIa and Q²-X is preferably on a ring atom that is not adjacent the bond to the remainder of the compound;
c) R² is of formula IIIa and Q¹ is a single bond;
d) R² is of formula IIIa and Q¹ is —CH═CH—;
e) R² is of formula IIIb, and R^{C1}, R^{C2} and R^{C3} are independently selected from H and methyl;
f) R² is of formula III, and Q is NR^{N}—R^{L1} optionally wherein R^{N} is H or methyl;
g) R² is of formula III, and Q is O—R^{L1} or S—R^{L1}.

92. A conjugate according to claim 91, wherein Q² is a single bond or Z—(CH₂)ₙ—, where Z may is O or S and n is 1 or 2.

93. A conjugate according to claim 87, wherein X is selected from the group consisting of: O—R^{L1}, S—R^{L1}, CO₂—R^{L1}, NH—C(═O)—R^{L1} and NR^{N}R^{L1}.

94. A conjugate according to claim 87, wherein R^{2′}, R^{6′}, R^{7′}, R^{9′}, R³⁰, R³¹ and Y′ are the same as R², R⁶, R⁷, R⁹, R¹⁰, R¹¹ and Y respectively.

95. A conjugate according to claim 87, which is of formula Ia, Ib or Ic: 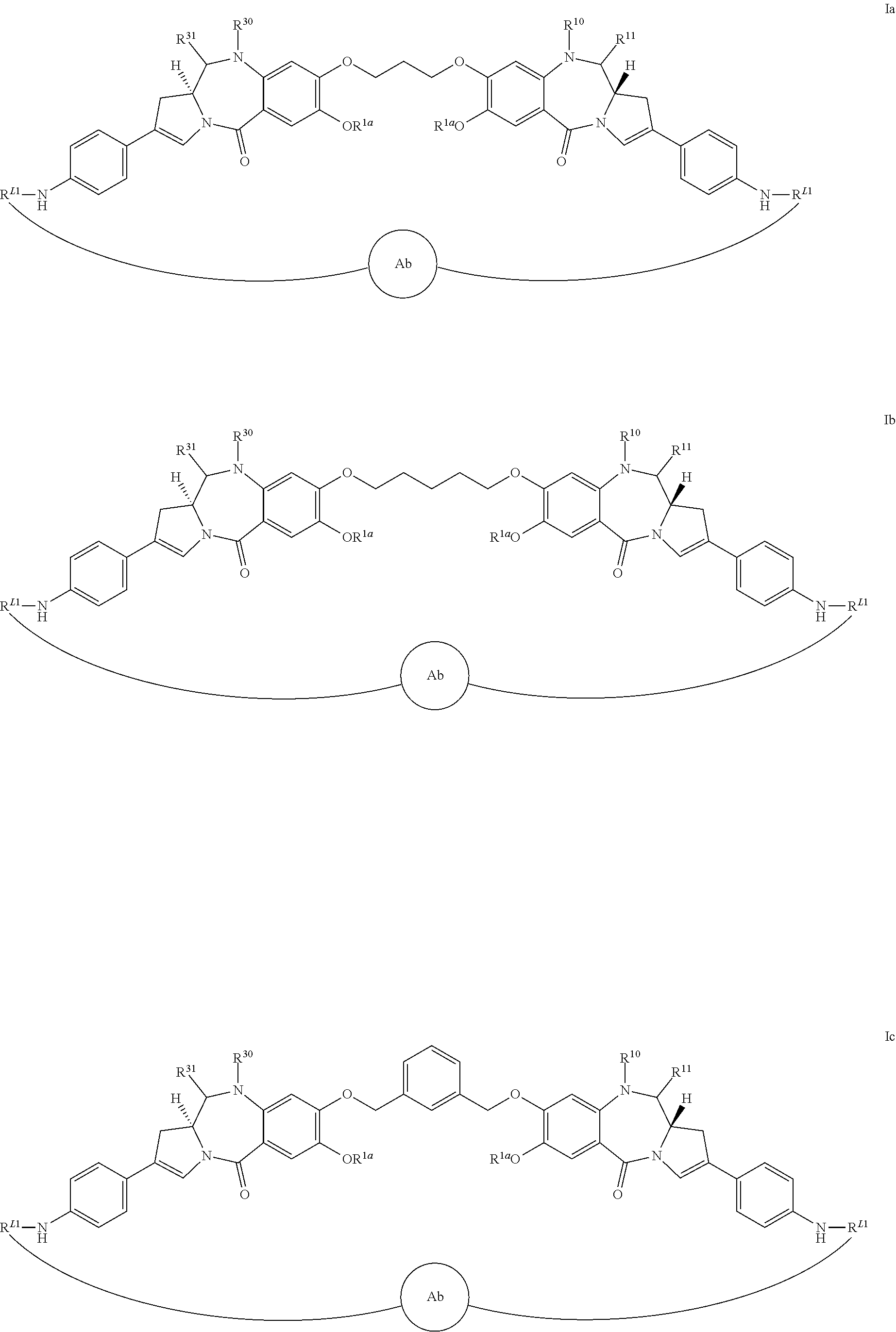
where
R^{1a} is selected from methyl and benzyl.

96. A conjugate of claim 87, wherein R^{L1} is Q^{X}-Z-G^{LL},
wherein
Q^{X} is selected from the group consisting of an amino acid residue, a dipeptide residue and a tripeptide residue,
Z is 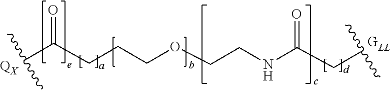
where a=0 to 5, b=0 to 16, c=0 or 1, d=0 to 5 and e is 0 or 1, and
G^{LL} is selected from: 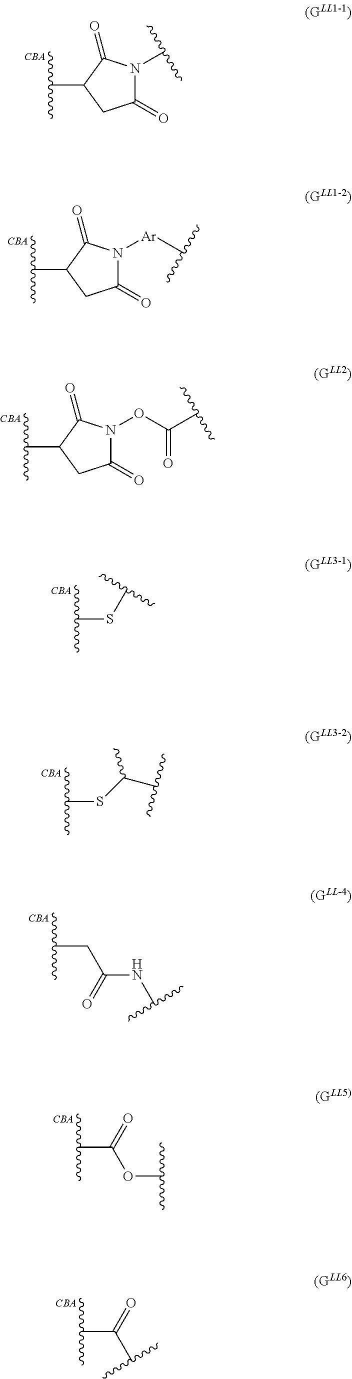 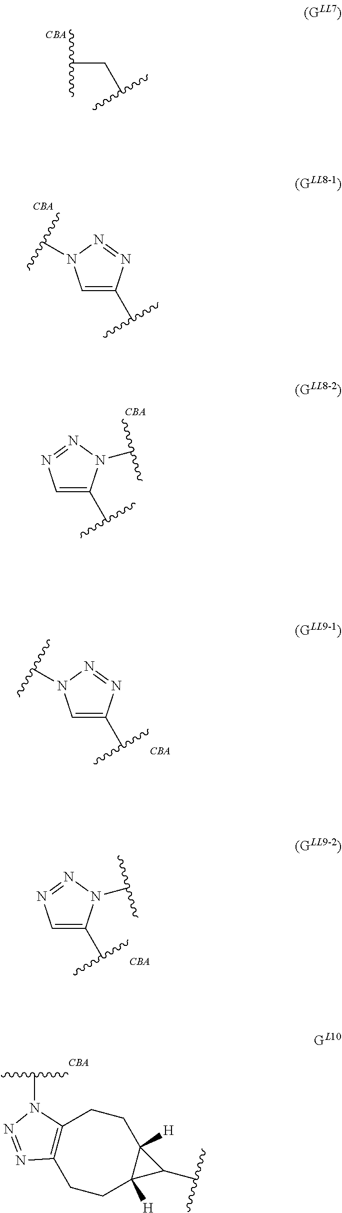 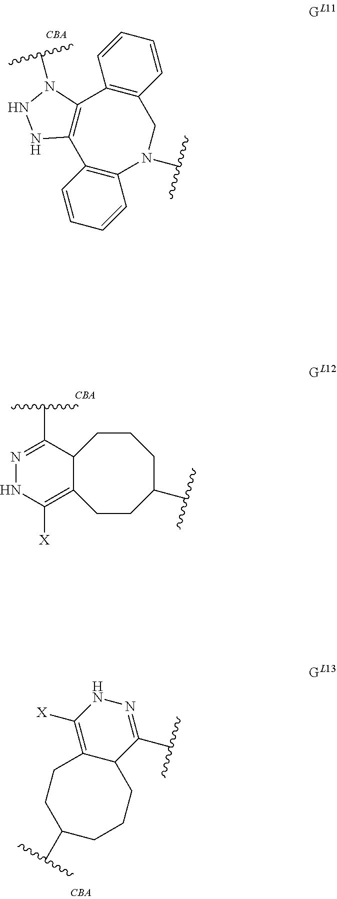
where Ar represents a C5-6 arylene group, e.g. phenylene and X represents C¹⁻⁴ alkyl.

97. A conjugate according to claim 96, wherein Q^{x} is:
a) an amino acid residue selected from Phe, Lys, Val, Ala, Cit, Leu, Ile, Arg, and Trp;
b) a dipeptide residue selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{NH},
^{CO}-Phe-Cit-^{NH},
^{CO}-Leu-Cit-^{NH},
^{CO}-Ile-Cit-^{NH},
^{CO}-Phe-Arg-^{NH}, and
^{CO}-Trp-Cit-^{NH};
c) a tripeptide residue.

98. A conjugate according to claim 87, wherein:
a) a is 0 to 3;
b) b is 0 to 12;
c) d is 0 to 3;
d) a is 0, b is 0, c is 0, d is 5 and e is 1;
e) c is 1, d is 2, e is 1, and b may be from 0 to 8.

99. A conjugate according to claim 87, wherein Ar is a phenylene group.

100. A conjugate according to claim 87 of formula Id: 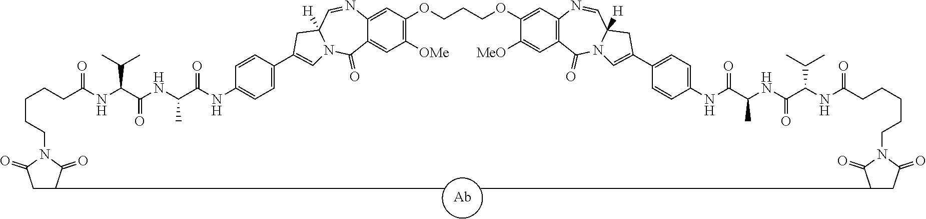 [SMILES: COc1cc2c(cc1OCCCOc1cc3c(cc1OC)C(=O)N1C=C(c4ccc(NC(=O)[C@H](C)NC(=O)[C@@H](NC(=O)CCCCCN5C(=O)CC(C)C5=O)C(C)C)cc4)C[C@H]1C=N3)N=C[C@@H]1CC(c3ccc(NC(=O)[C@H](C)NC(=O)[C@@H](NC(=O)CCCCCN4C(=O)CC(C)C4=O)C(C)C)cc3)=CN1C2=O]
or of formula Ie: 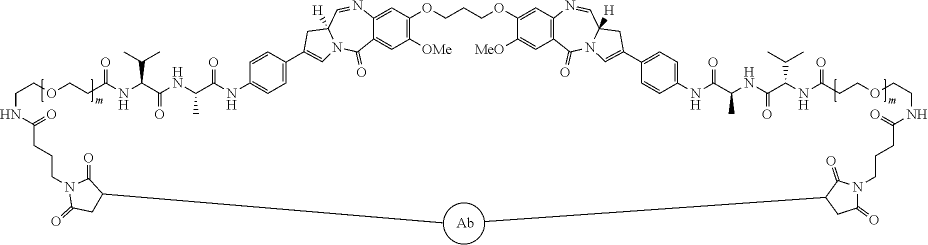
where m is an integer from 2 to 8.

101. The conjugate according to claim 87, wherein the modified antibody having at least one free conjugation site on each heavy chain is:
a) an IgG1, IgG2, IgG3 or IgG4 antibody;
b) is a human antibody; or
c) a humanized antibody.

102. The conjugate according to claim 101, wherein the native interchain cysteine residues have been substituted for amino acid residues lacking thiol groups.

103. The conjugate according to claim 102, comprising at least one additional substitutions in each heavy chain of an amino acid residue comprising a reactive group suitable for conjugation to a linker optionally wherein the additionally substituted amino acid is a cysteine or a non-natural amino acid optionally wherein the position that is substituted is selected from those set forth below:

| Antibody | Isotype | IgG1 | IgG2 | IgG3 | IgG4 |
|---|---|---|---|---|---|
| Position | 239 | Ser | Ser | Ser | Ser |
| (Kabat | 282 | Val | Val | Val | Val |
| EU) and | 289 | Thr | Thr | Thr | Thr |
| Corresponding | 297 | Asn | Asn | Asn | Asn |
| Amino Acid | 312 | Asp | Asp | Asp | Asp |
| | 324 | Ser | Ser | Ser | Ser |
| | 330 | Ala | Ala | Ala | Ser |
| | 335 | Thr | Thr | Thr | Thr |
| | 337 | Ser | Ser | Ser | Ser |
| | 339 | Ala | Thr | Thr | Ala |
| | 356 | Glu | Glu | Glu | Glu |
| | 359 | Thr | Thr | Thr | Thr |
| | 361 | Asn | Asn | Asn | Asn |
| | 383 | Ser | Ser | Ser | Ser |
| | 384 | Asn | Asn | Ser | Asn |
| | 398 | Leu | Leu | Leu | Leu |
| | 400 | Ser | Ser | Ser | Ser |
| | 422 | Val | Val | Ile | Val |
| | 440 | Ser | Ser | Ser | Ser |
| | 442 | Ser | Ser | Ser | Ser |
| | | | | |
|---|---|---|---|---|
| SEQ ID NO: | 1 | 2 | 3 | 4 |

104. A pharmaceutical composition comprising the conjugate of claim 87 and pharmaceutically acceptable diluent, carrier or excipient.

105. A method of medical treatment comprising administering to a patient the pharmaceutical composition of claim 104.

106. The method of claim 105, wherein the method of medical treatment is for treating cancer.

107. The method of claim 106, wherein the patient is administered a chemotherapeutic agent, in combination with the conjugate.

108. A method of treating a mammal having a proliferative disease, comprising administering an effective amount of a conjugate according to claim 87.

109. A compound of formula II: 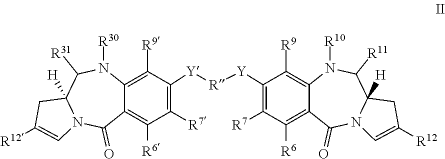
and salts and solvates thereof,
wherein R⁶, R⁷, R⁹, R¹⁰, R¹¹, Y, R″, Y′, R^{6′}, R^{7′}, R^{9′}, R³⁰ and R³¹ are as defined in any one of claims 87 to 94;
R¹² is of formula IVa, formula IVb or formula IVc: 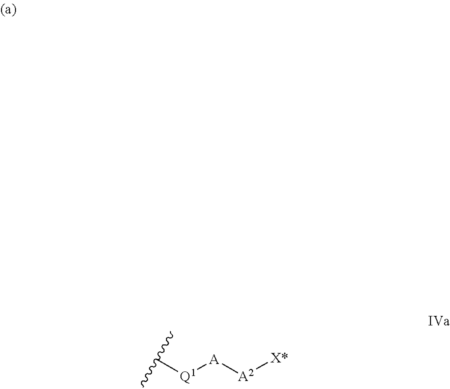
where A is a C₅₋₇ aryl group, and either
(i) Q¹ is a single bond, and Q² is selected from a single bond and —Z—(CH₂),ₙ—, where Z is selected from a single bond, O, S and NH and n is from 1 to 3; or
(ii) Q¹ is —CH═CH—, and Q² is a single bond; 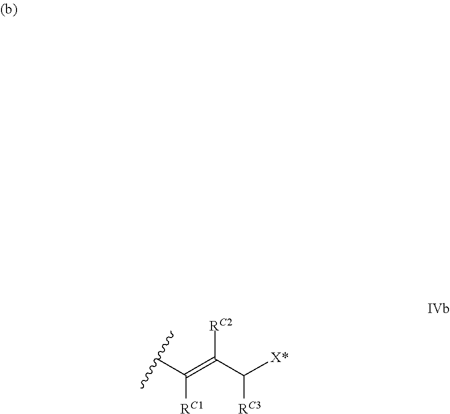
where;
R^{C1}, R^{C2} and R^{C3} are independently selected from H and unsubstituted C₁₋₂ alkyl; 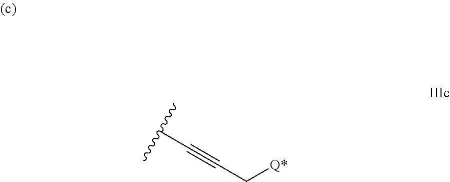
where Q* is selected from O—R^{G1}, S—R^{G1} and NR^{N}—R^{G1}, and R^{N} is selected from H, methyl and ethyl
X* is selected from the group comprising: O—R^{G1}, S—R^{G1}, CO₂—R^{G1}, CO—R^{G1}, NH—C(═O)—R^{G1}, NHNH—R^{G1}, CONHNH—R^{G1}, 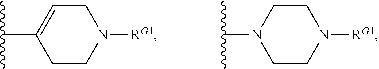
NR^{N}R^{G1}, wherein R^{N} is selected from the group comprising H and C₁₋₄ alkyl;
R^{G1} is Q^{X}-Z-G^{L},
wherein
Q^{X} is selected from the group consisting of an amino acid residue, a dipeptide residue and a tripeptide residue,
Z is 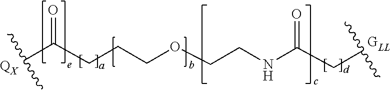
where a=0 to 5, b=0 to 16, c=0 or 1, d=0 to 5 and e is 0 or 1, and
G^{L} is selected from: 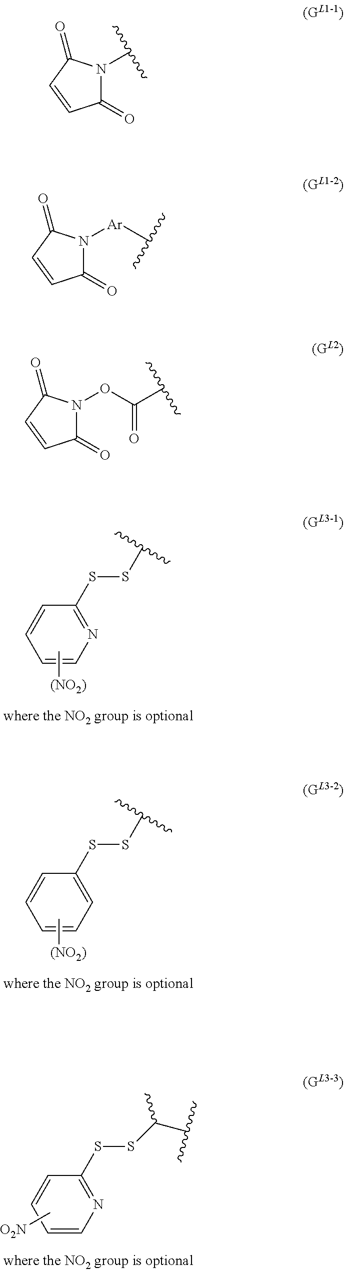 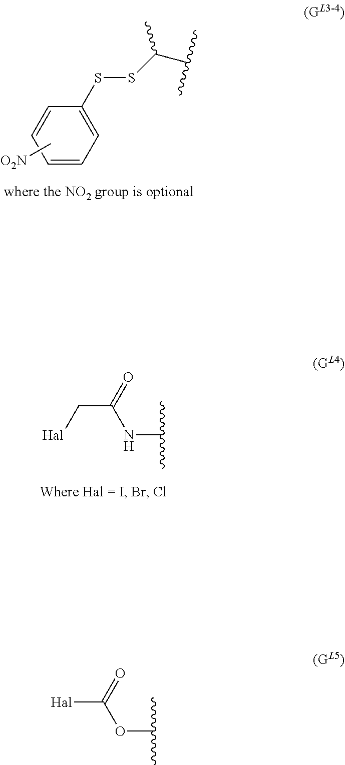 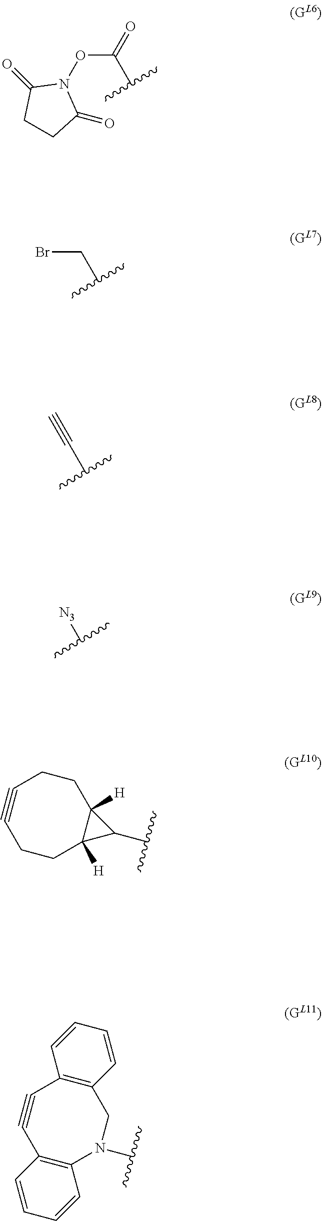 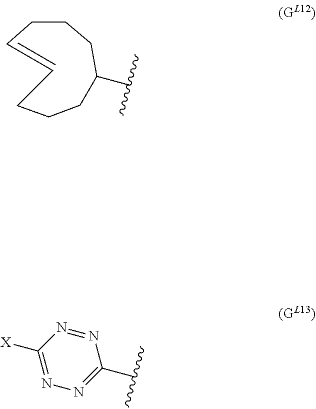
where Ar represents a C5-6 arylene group, e.g. phenylene, and X represents C¹⁻⁴ alkyl;
R^{12′} is selected from the same groups as R¹².

110. A compound according to claim 109, wherein Q^{x} is:
a) an amino acid residue selected from Phe, Lys, Val, Ala, Cit, Leu, Ile, Arg, and Trp;
b) a dipeptide residue selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{NH},
^{CO}-Phe-Cit-^{NH},
^{CO}-Leu-Cit-^{NH},
^{CO}-Ile-Cit-^{NH},
^{CO}-Phe-Arg-^{NH}, and
^{CO}-Trp-Cit-^{NH}; or
c) a tripeptide residue.

111. A compound according to claim 109, wherein:
a) a is 0 to 3;
b) b is 0 to 12;
c) d is 0 to 3;
d) a is 0, b is 0, c is 0, d is 5 and e is 1;
e) cis 1, d is 2, e is 1, and b may be from 0 to 8.

112. A compound according to claim 109, wherein Ar is a phenylene group.

113. A compound according to claim 109, wherein the compound is of formula Id′: 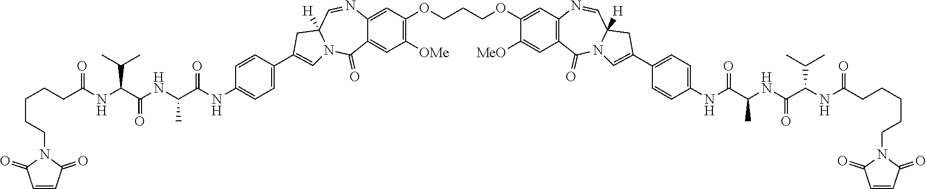 [SMILES: COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(NC(=O)[C@H](C)NC(=O)[C@@H](NC(=O)CCCCCN4C(=O)C=CC4=O)C(C)C)cc3)=CN1C2=O]
or formula (Ie′): 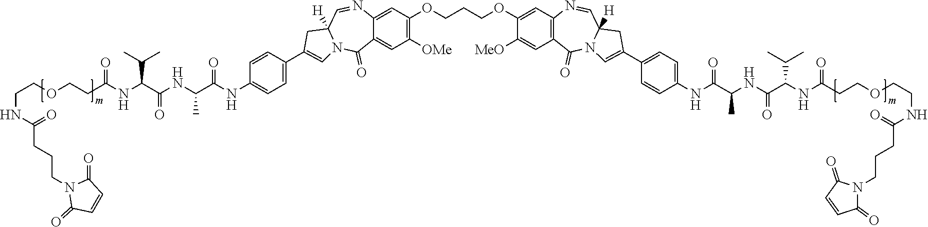
where m is an integer from 2 to 8.
